(19) United States
(12) Patent Application Publication
(10) Pub. No.: US 2023/0091170 A1
(43) Pub. Date: Mar. 23, 2023

(54) ENRICHMENT OF ANTIGEN-SPECIFIC ANTIBODIES FOR ANALYTIC AND THERAPEUTIC USE

(71) Applicant: Veravas, Inc., Oakdale, MN (US)

(72) Inventors: Joshua Caine Soldo, Prior Lake, MN (US); Scott Douglas Bergmann, River Falls, WI (US); Carmen L. Wiley, Spokane, WA (US)

(21) Appl. No.: 17/917,479

(22) PCT Filed: Apr. 12, 2021
(86) PCT No.: PCT/US2021/026927

§ 371 Date: Oct. 6, 2022

Related U.S. Application Data
(60) Provisional application No. 63/008,472, filed on Apr. 10, 2020.

(51) Int. Cl. G01N 33/68; C07K 16/06; G01N 33/58; C07K 16/42

(52) U.S. Cl. CPC G01N 33/6854; C07K 16/06; G01N 33/582; C07K 16/42

(57) ABSTRACT

The present invention is directed to methods for using particles (e.g, microparticulate, nanoparticulate; magnetic, non-magnetic) comprising surfaces comprising capture moieties as described herein, to remove an interference as described herein, or enrich biomarkers, especially antibodies, prior to a diagnostic test, or to be isolated and used for prophylactic or therapeutic purposes.

### CROSS-REFERENCE TO RELATED APPLICATIONS

This application claims the benefit of priority to U.S. Provisional Patent Application No. 63/008,472, filed Apr. 10, 2020, the entire contents of which are incorporate by reference herein.

### FIELD OF THE INVENTION

The present disclosure relates to methods for using particles (e.g, microparticulate, nanoparticulate; magnetic, non-magnetic) comprising surfaces comprising capture moieties as described herein to isolate antigen-specific antibodies for subsequent analytic, prophylactic, or therapeutic use.

### BACKGROUND

Laboratory testing plays a critical role in health assessment, health care, and ultimately the public's health, and affects persons in every life stage. Almost everyone will experience having one or more laboratory tests conducted during their lifetime. An estimated 7 to 10 billion laboratory tests are performed each year in the United States alone, and laboratory test results influence approximately 70% of medical decisions.

In addition, since the Centers for Medicare and Medicaid Services (CMS) on Jan. 1, 2018 implemented the new Clinical Laboratory Fee Schedule (CLFS) as required by the Protecting Access to Medicare Act (PAMA), PAMA is reducing lab testing reimbursement. It is even more critical lab results are accurate the first time and troubleshooting efforts are reduced or take less time and do not impact lab workflow.

Interference is a substance present in a patient specimen that can alter the correct value of the result of a diagnostic test, e.g., by interfering with antibody binding, or that can increase or decrease assay signal by bridging, steric hindrance, or autoantibody mechanisms. While it is known that immunoassays are susceptible to interference, the clinical laboratory may still report erroneous results if such results are not recognized and flagged by the instrument (analyzer) or laboratory, or if the physician does not notify the laboratory that the patient result does not fit the clinical picture. Erroneous results can occur unexpectedly with any specimen without the practical means to identify upfront such specimens likely to cause problems. The consequence of such interference is that erroneous results can result in false negatives and false positive test results, that can impact patient care, and can lead to unnecessary invasive, diagnostic or therapeutic procedures, or failure to treat a patient.

There are many sources of sample specific interference in the clinical laboratory such as sample type (i.e. plasma), carry-over, freeze/thaw, stability, hemolysis, icterus, lipemia, effects of anticoagulants, sample storage, binding proteins, drugs and drug metabolites, and cross-reactivity. However, heterophilic antibody interference such as human anti-animal antibody (HAAA) and human anti-mouse antibody (HAMA) interferences are troublesome and problematic as they are difficult to detect and can affect patient management.

Notwithstanding the complications arising from interference, biomarker screening and diagnostic testing can be difficult, for example because of their low presence or abundance in a biological sample.

Thus, while biomarkers found in the body can be used to detect, predict, or manage diseases, many are found in too low an abundance to be detected today using commercially available tests. There is an unmet clinical need for new diagnostic technology that prepares clinical samples to improve testing accuracy, measure hard to find biomarkers, reduce costs, and ultimately save lives.

Biotin, also known as vitamin B7, is a water-soluble B vitamin often found in multi-vitamins and over the counter health and beauty supplements. In vitro laboratory diagnostics tests that employ streptavidin-biotin binding mechanisms have the potential to be affected by high circulating biotin concentrations. Biotin can be attached through covalent bond to a variety of targets—from large antibodies to steroid hormones—with minimal effect on their specific non-covalent binding with avidin, streptavidin, or NeutrAvidin proteins. Therefore, biotin has been frequently used in the detection systems of immunoassays of different forms.

Immunoassays are generally categorized as either sandwich immunoassays (non-competitive) or competitive inhibition immunoassays. In general, streptavidin-biotin binding is used during assay incubation to couple biotinylated antibodies in sandwich immunoassays, or biotinylated antigens in competitive immunoassays, to streptavidin-coated surfaces. When a biological specimen contains excess biotin, the biotin competes with the biotinylated antibodies or antigens for binding to the streptavidin-coated surfaces, resulting in reduced capture of the biotinylated antibodies or antigens. Excess biotin produces falsely low results in sandwich immunoassays because the assay signal is directly proportional to the analyte concentration. Excess biotin in competitive immunoassays causes falsely elevated results because the assay signal is inversely proportional to the analyte concentration.

Normal circulating concentrations of biotin derived from the diet and normal metabolism are too low (<1.2 ng/mL) to interfere with biotinylated immunoassays. However, ingestion of high-dose biotin supplements (e.g., 5 mg or higher) can result in significantly elevated blood concentrations that can interfere with commonly used biotinylated immunoassays. Some studies have shown that serum concentrations of biotin can reach up to 355 ng/mL within the first hour after biotin ingestion for subjects consuming supplements of 20 mg biotin per day, and up to 1160 ng/mL for subjects after a single dose of 300 mg biotin. According to the FDA, biotin in blood or other samples taken from patients who are ingesting high levels of biotin can cause falsely high or falsely low results in biotin-based immunoassays, depending on the design of the assay..

Biotin in blood or other samples taken from patients who are ingesting high levels of biotin can cause falsely high or falsely low results in biotin-based immunoassays, depending on the design of the assay. Incorrect test results may lead to inappropriate patient management as well as misdiagnosis.

Biotin interference thresholds differ widely among assays, even on a single platform. Tests with biotin interference thresholds <51 ng/mL are considered high risk tests, or vulnerable immunometric and competitive methods.

Biotin-based tests are also susceptible to interference mechanisms associated with the use of streptavidin in the test design to capture biotin which has been conjugated to antibodies, proteins or antigens, or anti-streptavidin interference. Anti-streptavidin antibodies & proteins can significantly interfere with certain lab tests and cause incorrect test results. Similar to biotin interference which causes a decreased test signal and false low or false high patient results depending on the assay design and format, anti-streptavidin interference also results in a decreased test signal but via a different mechanism, and therefore it can be mistaken for biotin interference. Although the cause of anti-streptavidin antibodies is not fully known and under debate, one possible cause could be from the bacterium S. avidinii. It is thought that many people are exposed to these bacteria in daily life and could develop an immunological reaction to it.

There is therefore a clinical need for simple, inexpensive, automatable and effective solutions to eliminate or minimize sample interference and enrich biomarker concentration prior to diagnostic testing without impacting laboratory workflow and turnaround time.

### SUMMARY OF THE INVENTION

Described herein are methods for the simple, efficient and cost-effective conditioning of biological samples to manage and mitigate a multitude of known sample-specific interferences that can lead to erroneous test results and increased risk to patient safety, such as heterophilic antibodies in patients who have been treated with monoclonal mouse antibodies or have received them for diagnostic purposes. The methods described herein can also manage and mitigate sample-specific interferences that arise from biotin that can come from over the counter (OTC) supplements, multivitamins and herbal remedies taken by consumers for health & beauty and weight loss or therapeutically, e.g., for the treatment of multiple sclerosis.

Also described herein are methods for enriching or increasing the concentration of a biomarker in a biological sample. In particular, the biomarker may be antigen-specific antibody, for example a viral structural protein, such as the spike protein of Severe acute respiratory syndrome coronavirus 2 (SARS-CoV-2). In various embodiments the spike protein is the complete protein, the S1 subunit, the S2 subunit, or a antigenic fragment thereof, for example, a receptor binding domain (such as amino acids 331-524) and/or the N-terminal domain of the S1 subunit (such as, amino acid residues 1-260). The spike protein or antigenic fragment thereof can be biotinylated and attached to streptavidinated beads which can then serve as a capture reagent for SARS-CoV-2 neutralizing antibodies. Typically, if both the N-terminal domain and receptor binding domain are both used, there fragments are attached to separate beads, which are then mixed to serve as the capture reagent.

In an aspect, provided herein is a method for isolating antigen-specific antibody from a biological sample, the method comprising: a) combining the sample with a particle comprising a capture moiety to provide a mixture; and b) mixing the mixture to provide particle complexes to the antibody; thereby isolating the antibody from the biological sample. In some embodiments the capture moiety is the spike protein of SARS-CoV-2. In some embodiments, the capture moiety is the S1 subunit of the spike protein of SARS-CoV-2, or a receptor binding domain and/or an N-terminal domain thereof. The structure of the SARS-CoV-2 spike protein is known in the art (Walls et al, Structure, Function, and Antigenicity of the SARS-CoV-2 Spike Glycoprotein, Cell 180:1-12, 2020, which is incorporated herein by reference in its entirety). In various embodiments the biological sample can be blood, plasma, serum, or other antibody-containing biological fluids.

In some embodiment, the isolated antibody is detected, quantitated, or otherwise characterized in a serology assay.

In some embodiments the capture moiety-antibody complex is cleaved from the particle. In other embodiments the antibody is eluted from the capture moiety, especially while the capture moiety is still attached to the particle. The enriched or isolated antibody can then be subjected to protein chemistry analytic methods including mass spectrometry and Edman degradation. The enriched or isolated antibody can be used for passive immunization, for prophylactic or therapeutic purposes. For example, if antibodies recognizing the spike protein of SARS-CoV-2 are isolated, they can be administered as a therapeutic to a COVID-19 patient, or alternatively, they can be administered prophylactically to a healthcare worker or other person at risk of infection by SAR-CoV-2, due to exposure to COVID-19 patients.

In an aspect, provided herein is a method for removing an interference from a biological sample, the method comprising: a) combining the sample with a particle comprising a capture moiety to provide a mixture; b) mixing the mixture to provide particle complexes to the interference; and c) removing or eliminating the particle complexes to provide a depleted solution; thereby decreasing or reducing the amount (e.g., mass, molarity, concentration) of the interference.

### BRIEF DESCRIPTION OF THE DRAWINGS

FIG. 1 depicts a scheme for confirmation and disqualification assays based on removal (or depletion) of interferences from a biological sample by particles described herein.

FIG. 2 depicts a scheme for depletion assays based on removal (or depletion) of interferences from a biological sample by lyophilized particles described herein.

FIG. 3 depicts a scheme for depletion assays based on removal (or depletion) of interferences from a biological sample by magnetized pipette tips described herein.

FIG. 4 is a graph showing biotin concentration over time after biotin ingestion.

FIG. 5 is a graph showing biotin depletion.

FIG. 6 is a graph showing biotin depletion.

FIG. 7 is a graph showing biotin concentration over time after biotin ingestion.

FIG. 8 is a graph showing biotin concentration after ingestion of different biotin doses.

FIG. 9 is a graph showing biotin depletion.

FIG. 10 is a graph showing PTH concentration.

FIG. 11 depicts calibration curves for IgA, IgG, and IgM generated with triplex calibrator beads.

FIG. 12 presents total SARS-CoV-2 neutralizing antibody levels in 5 PCR-positive patients from which serial samples were available, who initially tested negative in the SARS-CoV-2 neutralizing antibody assay.

FIG. 13 presents total SARS-CoV-2 neutralizing antibody levels in 37 PCR-positive patients from which serial samples were available.

### DETAILED DESCRIPTION OF THE INVENTION

Described herein are methods for depleting or enriching a biological sample, the method comprising combining particles as described herein with a biological sample as described herein.

In an aspect, provided herein is a method for isolating a biomarker from a biological sample, the method comprising: a) combining the sample with a particle comprising a capture moiety to provide a mixture; and b) mixing the mixture to provide particle complexes to the biomarker; thereby isolating the biomarker from the biological sample. In some embodiments, the biomarker is antigen specific antibody. In some embodiments the antigen-specific antibody recognizes a spike protein of SARS-Cov-2 spike protein, for example the S1 subunit, or a receptor binding domain and/or an N-terminal domain thereof.

In some embodiments, the method further comprises subjecting the particle complexes to diagnostic testing. In some embodiments, the method further comprises subjecting biomarker cleaved or eluted from the particle complexes to diagnostic testing. In some embodiments, the biomarker is a pathogen-specific antibody. In some embodiments, the pathogen-specific antibody is anti-SARS-CoV2 antibody. In some embodiments, the anti-SARS-CoV-2 antibody comprises antibody recognizing the receptor binding domain, the N-terminal domain, or both.

There are SARS-CoV-2 neutralizing antibodies directed against both the receptor binding domain and the N-terminal domain. Antibodies binding to either of these domains sterically block the interaction of the S1 spike with the viral receptor (angiotensin converting enzyme 2 (ACE2)). Accordingly, antibodies binding these domains are considered neutralizing antibodies. IgM, IgG, and IgA isotype antibodies that recognize either of these domains are all considered to be neutralizing. Thus, to accurately assess the extent of neutralizing antibody in a biologic sample, it can be advantageous to capture and quantitate both kinds of antibody. Similarly, if the captured antibody is to be put to prophylactic or therapeutic use, more robust passive immunity can be established by capturing and used both kinds. For example, if antibodies recognizing both the receptor binding domain and the N-terminal domain are used a variant virus with a mutation in one of the domains will be less likely to escape neutralization than if antibody recognizing one of these domains were used.

To capture SARS-CoV-2 neutralizing antibodies, the SARS-CoV-2 S1-RBD and S1-NTD antigens are used in the capture reagent. In some embodiments, these antigens are biotinylated and coated on streptavidinated magnetic beads.

In an aspect, provided herein is a method for removing an interference from a biological sample, the method comprising: a) combining the sample with a particle comprising a capture moiety to provide a mixture; b) mixing the mixture to provide particle complexes to the interference; and c) removing or eliminating the particle complexes to provide a depleted solution; thereby decreasing or reducing the amount (e.g., mass, molarity, concentration) of the interference.

In some embodiments, the method further comprises subjecting the depleted solution to characterization (e.g., a diagnostic test).

In some embodiments, the particle is provided as a lyophized product (e.g., a LyoSphere™ (BIOLYPH LLC)).

In an aspect, provided herein is a method for increasing the accuracy of a diagnostic test, the method comprising: a) combining a biological sample with a particle comprising a capture moiety to provide a mixture; b) mixing the mixture to provide particle complexes to the interference; c) removing or eliminating the particle complexes to provide a depleted solution; and d) subjecting the depleted solution to the diagnostic test; thereby increasing the accuracy of the diagnostic test.

In some embodiments, at least 1%, 3%, 5%, 10%, 20%, 30%, 40%, 50%, 60%, 70%, 80%, 90%, 99% of the interference is removed in comparison to a biological sample not subjected to the method. In some embodiments, a sufficient amount of interference is removed to provide less than 100 ppm interference in the biological sample. In some embodiments, a sufficient amount of interference is removed to provide a less than detectable amount of the interference in a diagnostic test.

In some embodiments, the capture moiety is a human anti-animal antibody (e.g., mouse IgG, sheep IgG, goat IgG, rabbit IgG, cow IgG, pig IgG, horse IgG). In some embodiments, the capture moiety is a heterophilic antibody (e.g., FR (Fc-specific), Fab, F(ab)′2, polymerized IgG (type 1, 2a, 2b IgG and IgG fragments, serum components). In some embodiments, the capture moiety is an assay specific binder (e.g., biotin, fluorescein, anti-fluorescein poly/Mab, anti-biotin poly/Mab, streptavidin, neutravidin). In some embodiments, the capture moiety is an assay specific signal molecule (e.g., HRP, ALP, acridinium ester, isoluminol/luminol, ruthenium, N-(4-aminobutyl)-N-ethylisoluminol (ABEI)/cyclic ABEI). In some embodiments, the capture moiety is an assay specific blocker (e.g., BSA, fish skin gelatin, casein, ovalbumin, PVP, PVA). In some embodiments, the capture moiety is an assay specific conjugate linker (e.g., LC, LC-LC, PEO4, PEO16). In some embodiments, the capture moiety is an antigen autoantibody (e.g., free T3, free T4). In some embodiments, the capture moiety is a protein autoantibody (e.g., MTSH, TnI, TnT, non-cardiac TnT (skeletal muscle disease)). In some embodiments, the capture moiety is a chemiluminescent substrate (e.g., luminol, isoluminol, isoluminol derivatives, ABEI, ABEI derivatives, ruthenium, acridinium ester) or fluorescent label (e.g., fluorescein or other fluorophores and dyes). In some embodiments, the capture moiety is streptavidin, neutravidin, avidin, polyA, polyDT, aptamers, antibodies, Fab, F(ab′)2, antibody fragments, recombinant proteins, enzymes, proteins, biomolecules, or polymers. In some embodiments, the capture moiety is biotin, fluorescein, Po1yDT, PolyA, antigen, etc.

In some embodiments, the removing or eliminating is a separation. In some embodiments, the separation comprises physical separation. In some embodiments, the separation comprises magnetic separation. In some embodiments, the magnet for the magnetic separation is a multiple magnet device containing 2 to 12 magnets in a rack designed to hold 1 to 12 sample preparation tubes on a large pipetting machine. Examples of such pipetting machines include, but are not limited to, those built by Hamilton or Tecan. In some embodiments, the magnet for the magnetic separation is a multiple magnet device containing 96 or 384 magnets designed to provide magnetization to a 96 well or 384 well microtiter plate. In some embodiments, the separation comprises chemical separation. In some embodiments, the removing or eliminating comprises centrifugation at 1000×g or greater for at least 1 minute, 2 minutes, 3 minutes, 4 minutes, or 5 minutes to provide a pellet and a supernatant; and removing the supernatant. In some embodiments, the removing or eliminating comprises filtration (e.g., through a filter. In some embodiments, the filter has porosity or molecular weight cut-off (MWCO) sufficiently smaller than the diameter of the particle (e.g., nanoparticle, microparticle). In some embodiments, the filtration is by gravity, vacuum, or centrifuge. In some embodiments, the removing or eliminating comprises magnetization. In some embodiments, the magnetization occurs using a strong magnet (e.g., a neodymium magnet); to provide a pellet and a supernatant. In some embodiments, the magnet is in the centrifuge rotor. In some embodiments, the magnet is a magnet within a disposable pipette tip, cover or sheath.

In an aspect, provided herein is a method for isolating a biomarker from a biological sample, the method comprising: a) combining the sample with a particle comprising a capture moiety to provide a mixture; b) mixing the mixture to provide particle complexes comprising the biomarker; c) removing the particle complexes from the mixture; and d) adding to the mixture a cleavage reagent or releasing (elution) agent to provide an isolate comprising the biomarker; thereby isolating the biomarker from the biological sample. In some embodiments, the biological sample is pre-treated or cleaned, according to herein disclosed methods, prior to isolating the biomarker. In some embodiments, the biomarker is antigen-specific antibody. In some embodiments the antigen-specific antibody recognizes a spike protein of SARS-Cov-2 spike protein, for example the S1 subunit, or a receptor binding domain and/or an N-terminal domain thereof. In some embodiments, the method for isolating a biomarker from a biological sample is performed prior to performing a diagnostic test on the biological sample.

In some embodiments, the cleaning reagent includes human immunoglobulin, for example, IgG, IgA, and/or IgM as the capture moiety. In some embodiments, the cleaning reagent includes animal immunoglobulin, for example, rabbit, goat, or mouse IgG, as the capture moiety. In some embodiments, the cleaning reagent includes BSA. It is desirable to use the same microparticle used for the biomarker capture reagent in the cleaning reagent(s). In this manner, heterophilic interference(s) specific to the analyte antibody and assay reagents (for example, streptavidin and the beads themselves) can be removed.

In an aspect, provided herein is a method for determining whether a biomarker is present in a biological sample, the method comprising: a) combining the sample with a capture moiety to provide a mixture; b) combining the mixture with a particle comprising the capture moiety to provide a tertiary complex; c) removing the tertiary complex from the mixture to provide an isolate; and d) determining whether an indicator for the tertiary complex is present in the isolate; thereby determining whether the biomarker is present in a biological sample.

In an aspect, provided herein is a method for determining whether a biomarker is present in a biological sample, the method comprising: a) combining the sample with a particle comprising a capture moiety to provide a mixture; b) mixing the mixture to provide a particle complex to the interference; c) removing or eliminating the particle complexes to provide a depleted solution; d) combining the depleted solution with a second particle comprising a second capture moiety to provide a second mixture; e) mixing the second mixture to provide a second particle complex comprising the biomarker; f) removing the second particle complex from the second mixture; and g) adding to the second mixture a cleavage reagent or releasing agent to provide an isolate comprising the biomarker; thereby isolating the biomarker from the biological sample.

In some embodiments, the method further comprises washing the particle complex with a diluent.

In some embodiments, the cleavage reagent is a disulfide bond reducing reagent.

In some embodiments, the method further comprises performing a diagnostic test on the biomarker.

In an aspect, provided herein is a method for enriching an amount of a biomarker in a sample, the method comprising: a) adding to the sample a particle comprising a capture moiety to provide a mixture; b) mixing the mixture to provide a particle complex; c) separating the particle complex to provide a pellet and a supernatant; e) removing the supernatant from the pellet; f) washing the pellet with a diluent; and g) eluting the biomarker from the pellet to provide an enriched sample; thereby enriching the amount of a biomarker in the sample. In some embodiments, the method for enriching a biomarker from a biological sample is performed prior to performing a diagnostic test on the biological sample.

In some embodiments, the biomarker is an autoantibody against a tumor marker, for example, a tumor antigen, such as p53. In some embodiments, the tumor antigen is a neoantigen. Some tumor antigens are expressed in a developmentally inappropriate manner, for example, being expressed in a tissue or stage of maturation that it would not normally be expressed at all, or at as high level as it is being expressed. This can lead to the production of antibodies recognizing the tumor antigen. Other tumor antigens involved in the carcinogenic process may be mutated and the mutation is makes the tumor antigen immunogenic (a neoantigen). Again antibodies recognizing the altered tumor antigen can be produced. Detection of such autoantibodies can be useful in the detection and diagnosis of cancer, including early detection or changes in malignant state, and is useful in selecting appropriate treatment. Such tumor antigens can be used in the capture reagent for anti-tumor antigen autoantibodies.

Further autoantibodies against a tumor markers that can be useful for early detection of cancer include those recognizing Cancer Antigen 15-3 (CA15-3), carcinoembryonic antigen (CEA), Cancer Antigen 19-9 (CA19-9), c-Myc, p53, heat shock protein (Hsp)27 and Hsp70, eukaryotic translation initiation factor 3 subunit A (EIF3A), and Lung Cancer (LC). These tumor antigen-specific autoantibodies are promising biomarkers for early detection of cancer since they have long half-lives and are produced in large quantities in response to low circulating or low abundance cancer proteins.

In some embodiments, the biomarker is an indicator of traumatic brain injury (TBI). In some embodiments, the biomarker is s-100β, glial fibrillary acidic protein (GFAP), neuron-specific enolase (NSE), neurofilament light chain (NFL), cleaved tau protein (C-tau), and ubiquitin C-terminal hydrolase-L1 (UCH-L1). In some embodiments, the biomarker is an indicator of Alzheimer's Disease (AD). In some embodiments, the biomarker is amyloid beta, BACE1, soluble Aβ precursor protein (sAPP). In some embodiments, the biomarker is an indicator of a sexually transmitted disease (STD). In some embodiments, the STD is Chlamydia, Gonorrhea, Syphilis, Trichomonas, HPV, Herpes, Hepatitis B, Hepatitis C, HIV. In some embodiments, the biomarker is an indicator of bacterial infection. In some embodiments, the biomarker is capture moiety for a bacterium. In some embodiments, the biomarker is cleaved from the complex by a cleavage reagent. In some embodiments, the presence of biomarker is determined by MALDI-MS. In some embodiments, the presence of biomarker is determined by a molecular diagnostic method. In some embodiments, the presence of biomarker is determined by an immunoassay.

In some embodiments, the interference is fibrinogen and the removing or eliminating is separation, such as a physical separation by centrifugation, wherein the particle complexes are entrapped in a clot.

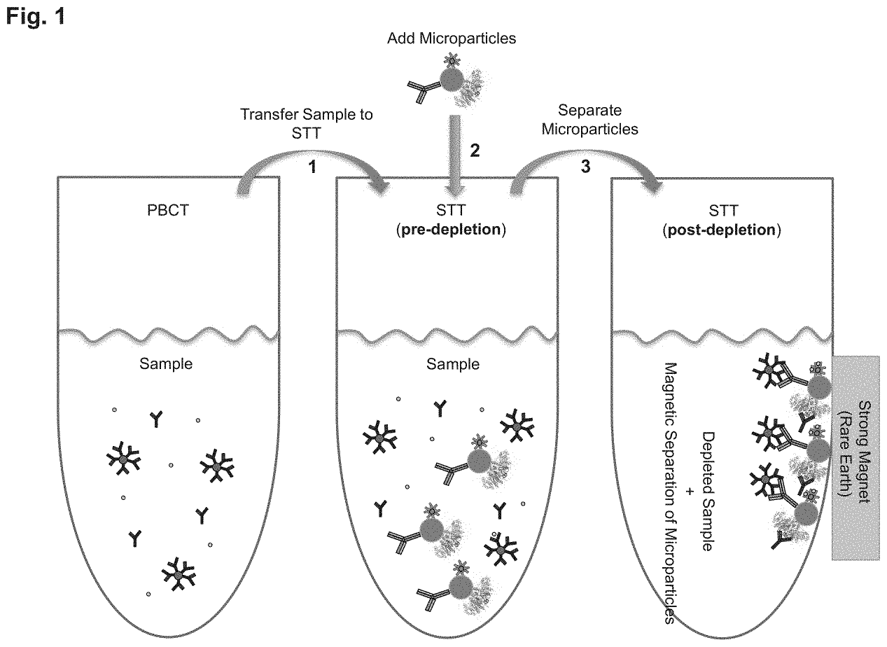

Turning to FIG. 1, a scheme is shown for confirmation and disqualification assays based on removal (or depletion) of interferences from a biological sample by particles described herein. A biological sample is aspirated from a primary blood collection tube (PBCT) and dispensed into the secondary transfer tube (STT). Particles described herein, e.g., particles comprising surfaces comprising capture moieties for free biotin and/or heterophilic antibodies are added to the STT to bind and deplete sample interferences.

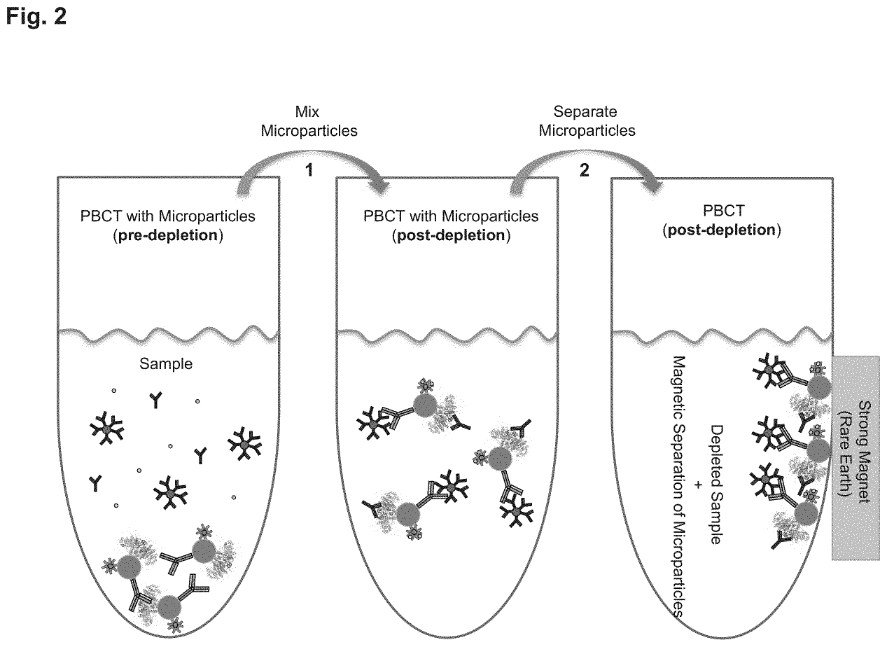

In FIG. 2, a scheme is shown for depletion assays based on removal (or depletion) of interferences from a biological sample by lyophilized particles described herein. A PBCT comprising lyophilized particles (e.g., particles as described herein) receive a biological sample, resulting in the resuspension and dispersement of particles with the biological sample.

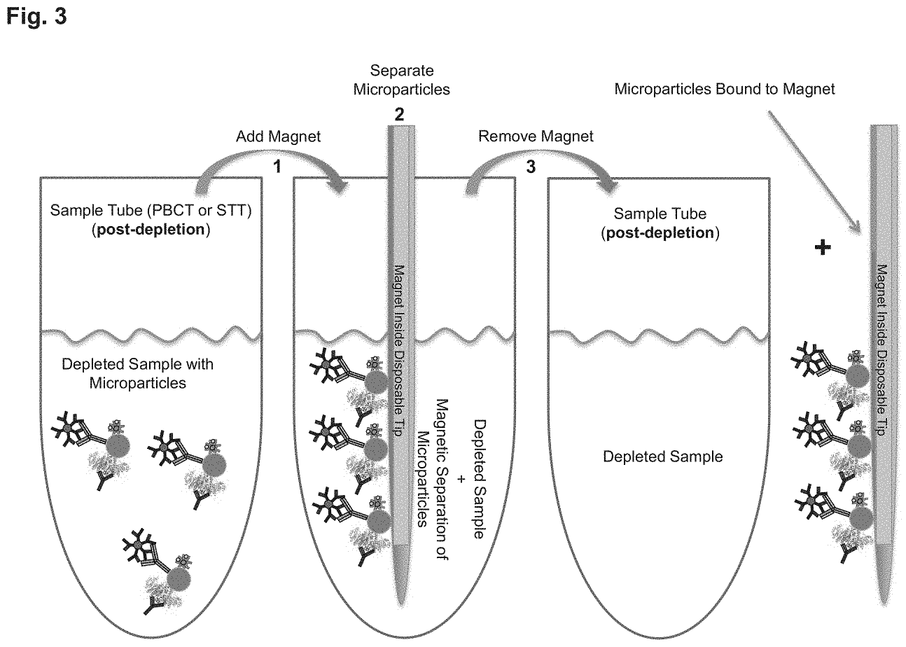

In FIG. 3, a scheme is shown for depletion assays based on removal (or depletion) of interferences from a biological sample by magnetized pipette tips described herein. A pipette tip comprising a magnet is added to a biological sample to remove from the biological sample an interference as described herein or biomarker as described herein.

### Method of Separation

Particles described herein can be added to a collection device such as a primary blood collection tube, 24-hr urine collection device, a urine collection device, a saliva collection tube, a stool collection device, a seminal fluid collection device, a blood collection bag, or any sample collection tube or device, prior to the addition of the biological sample.

Particles described herein can also be added to a sample after collection of the sample into a collection device, or after the transfer of the sample from a primary collection device into a storage or transfer device such as a plastic or glass tube, vial, bottle, beaker, flask, bag, can, microtiter plate, ELISA plate, 96-well plate, 384-well plate 1536 well plate, cuvette, reaction module, reservoir, or any container suitable to hold, store or process a liquid sample.

In some embodiments, the particles described herein are added to a collection device comprising a biological sample. In some embodiments, the particles described herein are added to a collection device prior to addition of a biological sample.

In some embodiments, especially embodiments involving preparative rather than analytic applications, biological samples from multiple donors are pooled prior to adding the particles. At least 10's of liters can be processed at a time.

In an aspect, described herein is a device for releasing particles comprising a collection device as described herein comprising a biological sample (i.e. screw cap which triggers release mechanism) such as on a urine collection device. For example, the device is a tube equipped with a screw cap that releases the particles described herein upon closure of the screw cap.

In an aspect, described herein is a device comprising a chemical release of particles to a container comprising a biological sample (i.e. encapsulated composition or composition that dissolves in solution at a defined rate or point in time). In some embodiments, the devices described herein are configured to delay the addition of particles described herein, for example to provide pre-treatment of sample prior to biomarker enrichment or isolation, or to diagnostic testing.

In some embodiments, the sample described herein can be pre-treated with a chemical, protein, blocker, surfactant or combination thereof prior to addition of the particles described herein for example to adjust pH, deplete or compete for sample specific interferences, and/or manage matrix specific challenges prior to the nanoparticles being added, introduced, dispersed or mixed in the sample to improve the specificity and binding kinetics of the nanoparticles to the target biomarker(s). The delayed addition of the nanoparticles to the sample after sample pre-treatment can he controlled physically by adding the nanoparticles to the sample after sample pre-treatment. The nanoparticles can also be present in the sample during the sample pre-treatment if the nanoparticles are encapsulated, shielded or protected by a chemical, polymer or sugar shell, coating, or polymerization such that the chemical, polymer or sugar needs to dissolve before the nanoparticles can be released, added, dispersed or mixed in the sample. The delayed release of nanoparticles can use chemistry known to someone skilled in the art such as used today in delayed drug release technology.

Preparative affinity separations have commonly used column chromatography. Magnetic particle separation technology can avoid problems of clogging of the column that some samples can cause. In one example, magnetic particle technology allows processing of whole blood or cell-containing blood fractions. Magnetic particle separation technology can also be accomplished in less time than a typical column-based affinity separation. Still another advantage of particle separation technology, is that elution of the captured ligand (biomarker) can be accomplished in a smaller volume, resulting in a more concentrated molecule without further processing.

### Methods of Magnetic Separation of Particles

In one aspect, provided herein is a method for removing an interference from a biological sample (e.g., prior to a diagnostic test, or prior to the enrichment or isolation of a biomarker), or to isolate or separate magnetic particle (e.g., within a primary blood collection tube, custom sample collection device, secondary transfer tube or custom sample device, or pooled samples). For example, a magnet-based device will quickly (less than 2 minutes; preferably less than 30 seconds) isolate the magnetic nanoparticles to the side(s) and/or bottom to form an essentially particle-free supernatant. The particle-free supernatant can be subsequently aspirated, drained, or otherwise removed without disrupting the pellet comprising the particles and dispensed into a separate transfer tube for diagnostic testing. In some embodiments, the pellet is isolated or subjected to diagnostic testing.

### Devices for the Magnetic Separation of Particles

Provided herein are devices comprising particles as described herein that can be used in the methods described herein to remove or deplete biomarkers, for example for diagnostic testing. In some embodiments, the devices comprise a physical mechanism to delay combination of the particles described herein with the samples described herein. In some embodiments, the devices described herein comprise a timed release mechanism to delay combination of the particles described herein with the samples described herein.

Magnetic Tube Holder. A custom magnetic tube holder, or a custom magnetic tube holder that can be removed from its stand, that can be inserted inside a sample rack for subsequent diagnostic testing of the particle-free supernatant. The custom magnetic tube holder can be designed to have physical openings or clear/transparent plastic (where magnets or the magnet array are not present) in its design where the sample tube barcode can still be detected and read by the analyzer, or where indices tests such as lipemia, hemolysis, cellular debris/clot detection, level sensing, etc. can still be performed by the analyzer. The sample tube could be a custom sample tube designed to have notches, or tongue and groove design, to only fit in the custom magnetic tube holder in a specific orientation to ensure the magnetic tube holder openings (space) or clear/transparent plastic allows the analyzer to see and read the barcode and/or perform indices tests such as lipemia, hemolysis, cellular debris/clot detection, level sensing, etc.

In some embodiments, the use of magnet(s) that can be attached to a sample rack via an adhesive, Velcro, or other method. Once the sample tube containing magnetic nanoparticles is inserted into the sample rack position(s) with magnet(s), the magnetic nanoparticles will quickly separate to the side(s) and/or bottom of the sample tube to form an essentially particle-free sample supernatant for diagnostic testing by the sample-rack specific testing platform or analyzer.

The sample rack itself as a custom magnetic sample rack compatible with a given analyzer (e.g., specific for the Abbott ARCHITECT, Siemens ADVIA Centaur XP, Roche cobas e411/e601/602/e801, Beckman Coulter Access 2/Dxl 400/Dxl 800, DiaSorin LIAISON/LIAISON XL, etc.). For example, every tube position in the rack will have an array of magnets designed to quickly separate the magnetic nanoparticles to the side(s) and/or bottom of the sample tubes to form essentially particle-free sample supernatants for diagnostic testing.

In an aspect, provided herein is a device (e.g., separation device) comprising a holder (e.g., a test tube holder) for a rack of test tubes, wherein the holder comprises a magnet.

Disposable Pipette Tip. In an aspect, the device is a disposable pipette tip comprising a custom magnet inserted inside the disposable tip to quickly isolate the magnetic nanoparticles to the surface of the pipette tip to form an essentially particle-free sample supernatant. The disposable pipette tip with custom magnet can subsequently be removed from the sample without disrupting the pellet comprising the particles. The disposable tip comprising particles can be discarded if there is no need to measure or characterize what the particles captured (i.e. interference depletion), or it can be inserted into a new tube for isolation and characterization of the particles in a subsequent diagnostic test (i.e., enrichment). For example, the disposable tip with particles can be inserted into a secondary transfer tube containing a buffer. If the magnet is removed from the tip, or if the magnet is turned off (e.g., electromagnet) the particles are free to disperse into the buffer.

In an aspect, provide herein is a device comprising a disposable pipette tip, wherein the tip comprises a magnet.

### Methods of Physical Separation of Particles

In one aspect, described herein are methods for removing particles described herein by physical force (e.g., gravitational force). In some embodiments, the particles described herein are separated, isolated, or removed (e.g., by centrifugation) from a biological sample by physical force. In some embodiments, the methods are used prior to application of diagnostic test methods described herein, for example, within a primary blood collection tube, custom sample collection device, secondary transfer tube or custom sample device. In some embodiments, the method for removing particles is filtration.

For example, magnetic nanoparticles specific for fibronectin and/or other clotting factors or off the clot components/constituents, cellular debris (i.e. red blood cell membrane specific) for the subsequent capture or binding of the “clot” (in serum) and/or capture or binding of cellular debris (in serum or plasma) enhance centrifugation speed and efficiency (shorter spin times to improve lab efficiency, workflow and throughput) by integration of strong magnets or magnetic technology in the centrifuge rotor and/or tube holders. This combination of RCF or Gs from centrifugation with magnetic separation of the magnetic nanoparticle complex (i.e. clot+magnetic beads, cellular debris+magnetic beads) enable much quicker and more efficient separation and supernatant formation on the side or bottom of the sample tube to clarify the sample for subsequent analysis. For example, this centrifugation step in most laboratories is 4 minutes or greater, and may be reduced to 2 minutes or less (preferable 1 min or less) by combining centrifugation with magnetic separation/isolation of the magnetic nanoparticle clot/cellular debris complexes.

Moreover, if the nanoparticles or plurality of magnetic nanoparticles are also specific for one or more different sample interference mechanisms such as 1, 5, 10, 20, 30, or more different interference mechanisms, these interferences, if present, would be captured by the nanoparticles and depleted from the sample after physical separation from centrifugation, or by the combination of centrifugation and magnetic separation described above.

While these magnetic nanoparticles do not need to also have specificity to the clot or cellular debris to be isolated via centrifugation or the combination of centrifugation and magnetic separation in the centrifuge, their surface could be co-coated or immobilized with more than one antibody and/or antigen where one or more antibodies would be specific for the clot and/or cellular debris, while the other antibody(s) and/or antigen would be specific to the sample interference. In this regard, the nanoparticles would specifically bind to both sample interference as well as the clot and/or cellular debris for subsequent physical separation or isolation via centrifugation or the combination of centrifugation and magnetic separation.

The use of nanoparticles specific for the clot and/or cellular debris increase clotting rate of speed by specific binding by the magnetic nanoparticles and pulling everything to a magnetic for magnetic separation and isolation. This bead-based pellet formed by the magnetic field and strength also accelerates the clot formation based on forced proximity of the clot or specifically captured clotting factors by the nanoparticles and subsequently the magnet.

### Methods of Chemical Separation of Particles

In some embodiments, the particles described herein are separated, isolated, or removed from a biological sample by chemical separation methods. In some embodiments, the chemical separation methods are used prior to application of diagnostic test methods, for example, within a primary blood collection tube, custom sample collection device, secondary transfer tube or custom sample device.

In one aspect, provided is a method for chemical separation of particles, the method comprising providing one or more of a salt, solvent, polymer, or detergent.

In some embodiments, the chemical separation methods, e.g., liquid-liquid phase separation will partition particles into a Phase A, and the nanoparticle free sample will be portioned into a Phase B where Phase B is tested. The agents for liquid-liquid phase separation (chemical phase separation) can be by salts, soluble polymers and detergents.

For example, liquid-liquid phase separation can occur by adding a non-polar solvent such as hexane to the polar aqueous sample where the particles partition into the non-polar phase leaving a nanoparticle-free aqueous phase for testing by a diagnostic test as described herein. In some embodiments, the method of separation described herein provide nanoparticles in the organic phase. In some embodiments, the method of separation described herein provide nanoparticles in the aqueous phase.

A method for isolating particles in a biological sample, the method comprising providing to the particles and biological sample a nonpolar solvent and an aqueous polar solvent to provide a nonpolar solvent layer and a polar solvent layer, removing a nonpolar solvent layer comprising the nonpolar solvent, and isolating the aqueous polar solvent comprising the particles, thereby isolating the particles.

Sample recovery can be adjusted or corrected by addition and use of an internal standard, such as a deuterated internal standard for LC-MS/MS, prior to aspirating and discarding the non-polar phase.

In some embodiments, the separation is physical separation used in combination with magnetic separation. For example, in an aspect, provided is a device (e.g., a magnetized centrifuge or a centrifuge equipped with a magnet that aids in separation by both the gravity and magnetic force of a magnet). In one aspect, provided herein is a device for separation of a particle described herein, the device comprising a magnet and centrifuge. In some embodiments, the device significantly reduces the time of centrifugation.

### Method for the Removal of Interferences

Described herein are methods for removing or minimizing interferences, the method comprising depleting (e.g, mitigating, reducing or managing) known pre-analytical and analytical sources of testing error (e.g., interferences) due to hemolysis, lipemia, icterus, bilirubin, microfibrin clots, cellular debris, blood cells, fibrinogen, other interfering substances such as drugs, metabolites, supplements, herbal remedies, and multivitamins. In some embodiments, the methods described herein provide methods for removing interference due to matrix effects or sample-type differences (e.g., animal species, human species). In some embodiments, the methods described herein provide methods for removing interferences prior to diagnostic testing (e.g., diagnostic or biomarker testing for example in a clinical trial). In some embodiments, the methods for removing biomarkers described herein are used in a clinical trial to improve the accuracy and dependability of a diagnostic test of a biomarker described herein. For example, the methods described herein can be used in patient selection or screening, e.g., for inclusion or exclusion criteria. In some embodiments, the methods or removal or depletion described herein can be used to identify outliers in clinical data or clinical trial results. For example, an outlier in clinical data or clinical trial results include a false positive or false negative identification for a biomarker described herein.

Depletion is defined as complete if sufficient quantity of interference is captured and/or removed for subsequent interference-free or reduced quantitative, semi-quantitative, or qualitative analysis. Depletion is defined as partial if sufficient quantity of interference(s) or interference mechanism(s) is captured and/or removed for subsequent semi-quantitative or qualitative analysis, or also partial if sufficient quantity of interference(s) or interference mechanism(s) and internal standard(s) is captured for quantitative, semi-quantitative or qualitative analysis by measurement methods that can use internal standards to adjust for recovery of the target analyte(s) or biomarker such as LCMS and LC-MS/MS (i.e. deuterated internal standard) and HPLC (C14 or tritiated internal radioisotope internal standards).

Depletion does not imply 100% removal of interference from the sample but means that residual interference no longer results in an erroneous result. However, sample pre-treatment depletion can result in 100% removal of interference if required for a particular assay or purpose such as subsequent elution and analysis by LC-MS/MS, or for sample preanalytical processing, nucleic acid purification and concentration for molecular diagnostics, or for the enrichment of biomarkers from challenging sample types such as urine, saliva and stool.

In some embodiments, the method s described herein is performed is less than 1 week, 6 days, 5 days, 4 days, 3 days, 2 days, 1 day, 24 hours, 20 hours, 16 hours, 12 hours, 8 hours, 4 hours, 2 hours, 1 hour, 30 minutes, 15 minutes, 10 minutes, 5 minutes or less. In some embodiments, the methods described herein is performed in less than 1 day.

### Interferences

The methods provided herein reduce, minimize, or eliminate an interference in a biological sample. Interference is a substance present in a patient specimen that can alter the correct value of the result of a diagnostic test, e.g., by interfering with antibody binding, or that can increase or decrease assay signal by bridging, steric hindrance, or autoantibody mechanisms. “Interferences,” as used herein, refers to any endogenous or exogenous substance or combination of endogenous and/or exogenous substances in blood, plasma, serum, CFS, urine, stool, saliva, semen, amniotic fluid, or other bodily fluids or sample matrices, such as immunoglobulins (IgG, IgM, IgA, IgE, IgD), proteins, antigens, lipids, triglycerides, cellular constituents, foreign substances, chemicals, drugs, drug metabolites, supplements, vitamins, herbal remedies, foreign bodies (viruses, bacterium (gram positive, gram negative), fungi, yeast) and waste products produced by any foreign bodies, food or dietary substances that can interfere with a test and result in an erroneous test results by specific or non-specific interactions with the test raw materials, formulation, biological and synthetic components, test design, and/or test format. Interferences can be, but not limited to, heterophile or heterophile-like interferences such as autoantibodies, rheumatoid factor (RF), human anti-mouse antibodies (HAMA), human anti-animal antibodies (HAAA) such as goat, rabbit, sheep, bovine, mouse, horse, pig, and donkey polyclonal and/or monoclonal antibodies, and manufacture assay-specific interference used in the test design or assay formulation, such as the chemiluminescent substrate (luminol, isoluminol, isoluminol derivatives, ABEI, ABEI derivatives, ruthenium, acridinium ester), fluorescent labels such as fluorescein or other fluorophores and dyes, capture moieties (streptavidin, neutravidin, avidin, CaptAvidin, polyA, polyDT, aptamers, antibodies, Fab, F(ab′)2, antibody fragments, recombinant proteins, enzymes, proteins, biomolecules, polymers) and their binding partners (i.e. biotin, fluorescein, Po1yDT, PolyA, antigen, etc.), conjugation linkers (LC, LC-LC, PEO, PEOn), bovine serum albumin, human serum albumin, ovalbumin, gelatin, purified poly- and monoclonal IgG such as mouse, goat, sheep and rabbit, polyvinyl alcohol (PAA), polyvinylpyrrolidone (PVP), Tween-20, Tween-80, Triton X-100, triblock copolymers such as Pluronic and Tetronic, and commercially available blockers, blocking proteins and polymer-based blocking reagents such those from Surmodics and Scantibodies) typically used in the design of antibody-based diagnostic tests, non-antibody based diagnostic tests, or sample pre-treatment methods and devices for subsequent analysis by mass spectrometry (i.e. HPLC, MS, LCMS, LC-MS/MS), radioimmunoassay (RIA), enzyme-linked immunoassay (ELISA), chemiluminescence immunoassay (CLIA), molecular diagnostics, lateral flow, point-of-care (PoC), CLIA and CLIA waived tests and devices.

In an aspect, provided herein is a method for removing from a biological sample an interference (e.g., biotin), the method comprising providing a particle derivatized with a capture moiety (that will bind to the inference). In some embodiments, the interference is biotin.

In another aspect, a sample can be pre-treated with a particle (e.g., nanoparticle, microparticle) to deplete sex hormone-binding globulin (SHBG) or sex steroid-binding globulin (SSBG) from serum or plasma such that the SHBG-depleted sample could be subsequently tested to measure free or bioavailable hormone or steroid (i.e. free testosterone). In some embodiments, the interference is sex hormone-binding globulin (SHBG) or sex steroid-binding globulin (SSBG).

In some embodiments, the interference is biotin, HAMA, RF, Heterophilic, or anti-SAv.

### Method for the Removal or Enrichment of Biomarkers

Described herein are methods for enriching or increasing the concentration of a biomarker in a biological sample. “Enrichment” is defined as the complete or partial particle capture and binding of target analyte(s) or biomarker to the particles from a biological sample (e.g., human or animal serum, plasma, blood, whole blood, processed blood, urine, saliva, stool (liquid and solid), semen or seminal fluid, cells, tissues, biopsy material, DNA, RNA, or any fluid or solid). In some embodiments, enrichment comprises washing and concentration of a biological sample, for example by allowing the biomarker-specific nanoparticles to be washed, then isolated to remove or minimize interferences prior to a biomarker characterization and measurement step.

In some embodiments, the methods described herein are used to isolate and purify a specific target (e.g., a biomarker) in a biological sample for subsequent elution and testing or other use, or to enrich or increase the concentration of the biomarker prior to a diagnostic test, further purification, formulation, or other use.

After washing or isolating the biomarker specific particles, the particles can be dispersed, reconstituted or resuspended in a buffer such as phosphate buffered saline (i.e. PBS pH 7.2), or LC-MS/MS compatible buffer, prior to the characterization or measurement step. This means the key characterization or measurement step of the captured and enriched biomarkers by the particles occurs in a buffer system and not in the animal or human matrix which is what introduces or causes the matrix effect or bias between biomarkers measured in animal blood, plasma, serum or urine as compared to the same biomarkers measured in blood, plasma, serum or urine using the same characterization, measurement or test method or system. Wash allows sample matrix, components, proteins and cellular constituents and associated interference or matrix effect be washed away. Similarly, the isolated biomarker may be removed from the animal or human matrix and released into a formulation buffer for therapeutic or prophylactic use.

Enrichment is defined as complete if sufficient quantity of anayte(s) is captured for subsequent diagnostic test, e.g., quantitative, semi-quantitative, or qualitative analysis, and is defined as partial if sufficient quantity of analyte(s) or biomarker is captured for subsequent semi-quantitative or qualitative analysis, or also partial if sufficient quantity of target analyte(s) or biomarker and internal standard(s) is captured for quantitative, semi-quantitative or qualitative analysis by measurement methods that can use internal standards to adjust for recovery of the target analyte(s) or biomarker such as LCMS and LC-MS/MS (i.e. deuterated internal standard) and HPLC (C14 or tritiated internal radioisotope internal standards). Enrichment is defined as preparative if sufficient quantity of the captured species is obtained for subsequent use in a prophylactic or therapeutic product.

A method is provided herein for enriching a biomarker in a sample prior to a diagnostic test consisting of: a) adding a particle (e.g., nanoparticle, microparticle) to the sample; b) mixing the sample with the particle (e.g., nanoparticle, microparticle); c) incubating the particle (e.g., nanoparticle, microparticle) with the sample to bind and capture the biomarker to the particle (e.g., nanoparticle, microparticle); d) separating or removing the particle (e.g., nanoparticle, microparticle) from the sample; e) saving the particle (e.g., nanoparticle, microparticle); f) washing the particle (e.g., nanoparticle, microparticle) using an appropriate wash diluent to remove non- specific materials; g) measuring the amount, mass, molarity, concentration, or yield of biomarker captured by the particle (e.g., nanoparticle, microparticle) using a qualitative, semi-quantitative or quantitative diagnostic test specific for the biomarker. In some embodiments, the diluent comprises water (e.g., deionized water, water for injection, saline, a buffered aqueous solution).

In some embodiments, the methods of enrichment described herein comprise a washing step. The washing step removes interferences as described herein and/or provides washed, purified, or isolated biomarker of interest (e.g., a biomarker as described herein). In some embodiments, the methods of enrichment described herein reduce matrix effects or species effects. In some embodiments, the methods of enrichment described herein are used prior to a diagnostic test comparing two biological samples of different origin. In some embodiments, the methods of enrichment described herein are used prior to a diagnostic test comparing an animal sample and a human sample. In some embodiments, the methods of enrichment described herein are used prior to a diagnostic test comparing a serum sample and a plasma sample. In some embodiments, the methods of enrichment described herein is used on a sample of high viscosity.

In some embodiments, the methods of enrichment comprise combining of a first biological sample enriched with a biomarker with a second biological sample enriched with the biomarker.

Provided herein is a method of measuring the amount, mass, molarity, concentration, or yield of targeted biomarker captured and enriched by the particle (e.g., nanoparticle, microparticle) whereby the biomarker is eluted, disassociated or freed from the particle (e.g., nanoparticle, microparticle) by the cleavage reagent described herein, e.g., by disrupting the binding interaction using elution strategies such as pH (e.g. increased pH with a base such as sodium bicarbonate, decreased pH with an acids such as acetic acid, trichloroacetic acid, sulfosalicylic acid, HCl, formic acid, and common pH elution buffers such as 100 mM glycine.HCl, pH 2.5-3.0, 100 mM citric acid, pH 3.0, 50-100 mM triethylamine or triethanolamine, pH 11.5, 150 mM ammonium hydroxide, pH 10.5), a displacer or displacing agent, competitive elution (e.g. >0.1M counter ligand or analog), ionic strength and/or chaotropic effects (e.g. NaCl, KCl, 3.5-4.0M magnesium chloride pH 7.0 in 10 mM Tris, 5M lithium chloride in 10 mM phosphate buffer pH 7.2, 2.5M sodium iodide pH 7.5, 0.2-3.0M sodium thiocyanate), surfactant, detergent, a concentrated inorganic salt, denaturing (e.g. 2-6M guanidine.HCl, 2-8M urea, 1% deoxycholate, 1% SDS), an organic solvent (e.g. alcohol, chloroform, ethanol, methanol, acetonitrile, hexane, DMSO, 10% dioxane, 50% ethylene glycol pH 8-11.5 (also chaotropic)), radiation or heat (increased temperature), conformational change, disulfide bond reducers (2-mercaptoethanol, dithiothreitol, tris(2-carboxylethyl)phosphine), enzyme inactivation, chaotropic agents (Urea, Guanidinium chloride, Lithium perchlorate), mechanical agitation, sonication, and protein digestive enzymes (pepsin, trypsin), and combinations thereof.

In some embodiments, 100 mM glycine, pH 2.5 is used as an elution buffer to release complexed anti-IgA, IgG, and/or IgM detection antibody (e.g., AlexaFluor488-anti-human IgG, AlexaFluor555-anti-human IgM, AlexaFluor647-anti-human IgA) or captured IgA, IgG, and/or IgM antibody complexed with a labeled detection antibody, from the capture beads. The magnetic beads are subsequently isolated with a strong magnetic and the eluate is then transferred to a new well with a neutralization buffer, for example, 300 mM Tris pH 10.0, to neutralize the pH and improve stability of the fluorophores for subsequent detection by a fluorimeter or fluorescent reader. This neutralization of the acidic elution pH can be important to improve assay precision and reproducibility.

Unless otherwise stated, or implicit from the disclosure, any of the embodiments described in connection with any particular method or composition described herein can be used in conjunction with any of the other embodiments described herein.

The methods and compositions of the various embodiments can be used in conjunction with any suitable assay known in the art, for example any suitable affinity assay or immunoassay known in the art including, but not limited to, protein-protein affinity assays, protein-ligand affinity assays, nucleic acid affinity assays, indirect fluorescent antibody assays (IFAS), enzyme-linked immunosorbant assays (ELISAs), radioimmunoassays (RIAs), and enzyme immunoassays (EIAs), direct or indirect assays, competitive assays, sandwich assays, CLIA or CLIA waved tests, LC-MS/MS, analytical assays, etc.

A method of both depleting sample interferences and enriching biomarkers from the same sample prior to the diagnostic test consisting of: a) add a chemical and/or biological reagent, additive or composition to the sample to block or deplete sample-specific interferences prior to the addition of a biomarker specific particle (e.g., nanoparticle, microparticle) to the sample; b) add a biomarker specific particle (e.g., nanoparticle, microparticle) to the sample after pre-treating or incubating the sample with the chemical and/or biological reagent, additive or composition; c) incubate the biomarker specific particle (e.g., nanoparticle, microparticle) with the sample to bind and capture the targeted biomarker(s) to the particle (e.g., nanoparticle, microparticle); d) wash the particle (e.g., nanoparticle, microparticle) or isolate it from the sample and chemical and/or biological reagent, additive or composition e) characterize the biomarker(s) captured and enriched by the particle (e.g., nanoparticle, microparticle) using a diagnostic test.

For example, in an embodiment, a particle bound to CaptAvidin would bind to biotin in a sample at neutral pH. The biotin bound to the CaptAvidin particle would release biotin when the pH is raised to 10.

### Biomarkers

Described herein are methods to isolate or for isolating or enriching a biomarker present in a biological sample. A “biomarker,” as referred to herein, is defined as a distinctive biological or biologically derived indicator (e.g., a metabolite) of a process, event, or condition such as aging or disease. Biomarkers may be an endogenous and/or exogenous analyte, antigen, small molecule, large molecule, drug, therapeutic agent, metabolite, xenobiotic, chemical, peptide, protein, protein digest, viral antigen, bacteria, cell, cell lysate, cell surface marker, epitope, antibody, a fragment of an antibody, IgG, IgM, IgA, IgE, IgD receptor, a ligand of a receptor, hormone, a receptor of a hormone, enzyme, a substrate of an enzyme, a single stranded oligonucleotide, a single stranded polynucleotide, a double stranded oligonucleotide, a double stranded polynucleotide, polymer and aptamer. In some embodiments, biomarkers is an interference described herein (e.g., a substance present in a patient specimen that can alter the correct value of the result of a diagnostic test, e.g., by interfering with antibody binding, or that can increase or decrease assay signal by bridging, steric hindrance, or autoantibody mechanisms. In some embodiments, the biomarker is an antibody to an infectious disease antigen, for example a viral antigen. The antibody to an infectious disease antigen can indicate exposure to, and recovery from, infection with the infectious disease agent. In such instances the antibody can be used for passive immunization for therapeutic or prophylactic purposes. In some embodiments the antibody to an infectious disease antigen recognizes a spike protein of SARS-Cov-2 spike protein, for example the S1 subunit, or a receptor binding domain and/or an N-terminal domain thereof. “Interferences,” as used herein, can be, but not limited to, heterophile or heterophile-like interferences such as autoantibodies, rheumatoid factor (RF), human anti-mouse antibodies (HAMA), human anti-animal antibodies (HAAA) such as goat, rabbit, sheep, bovine, mouse, horse, pig, and donkey polyclonal and/or monoclonal antibodies, and manufacture assay-specific interference used in the test design or assay formulation, such as the chemiluminescent substrate (luminol, isoluminol, isoluminol derivatives, ABEI, ABEI derivatives, ruthenium, acridinium ester), fluorescent labels such as fluorescein or other fluorophores and dyes, capture moieties (streptavidin, neutravidin, avidin, polyA, polyDT, aptamers, antibodies, Fab, F(ab′)2, antibody fragments, recombinant proteins, enzymes, proteins, biomolecules, polymers) and their binding partners (i.e. biotin, fluorescein, Po1yDT, PolyA, antigen, etc.), conjugation linkers (LC, LC-LC, PEO, PEOn), bovine serum albumin, human serum albumin, ovalbumin, gelatin, purified poly- and monoclonal IgG such as mouse, goat, sheep and rabbit, polyvinyl alcohol (PAA), polyvinylpyrrolidone (PVP), Tween-20, Tween-80, Triton X-100, triblock copolymers such as Pluronic and Tetronic, and commercially available blockers, blocking proteins and polymer-based blocking reagents such those from Surmodics and Scantibodies) typically used in the design of antibody-based diagnostic tests, non-antibody based diagnostic tests, or sample pre-treatment methods and devices for subsequent analysis by mass spectrometry (i.e. HPLC, MS, LCMS, LC-MS/MS), radioimmunoassay (RIA), enzyme-linked immunoassay (ELISA), chemiluminescence immunoassay (CLIA), molecular diagnostics, lateral flow, point-of-care (PoC), CLIA and CLIA waived tests and devices). In some embodiments, biomarkers are found in biological samples described herein.

Fibrinogen. Fibrinogen is converted during tissue and vascular injury by thrombin to fibrin, which subsequently results in the formation of a fibrin-based blood clot. In some embodiments, the particles described herein (e.g., particle-derivizatized anti-fibrinogen (e.g., mouse anti-fibrinogen)) used in the methods described herein bind and allow separation (e.g., chemical separation) of fibrinogen in whole blood. Particle binding to the clot via fibrin can be isolated and removed from the serum post-centrifugation for particle-free serum testing. In some embodiments, the biomarker is fibrinogen. In some embodiments, the methods described herein use particle-derivizatized anti-fibrinogen to remove the need for centrifugation of samples (e.g., blood samples).

Traumatic Brain Injury. In one embodiment, the biomarker is for traumatic brain injury. There are nine (9) biomarkers associated with the severity and magnitude of acute brain injury and the integrity of the blood brain barrier (BBB), but they are present at very low circulating concentrations in blood and are very difficult to detect and quantitate using existing immunoassay technologies and test platforms. While the Banyan BTI test (FDA cleared Feb. 14, 2018) measures only 2 of these biomarkers, the methods and devices (e.g., methods of enrichment; devices for enrichment) described herein enable simultaneous measurement of all 9 biomarkers in a patient to aid in the near patient diagnosis and prognosis. Particles derivatized with capture moieties for each of the 9 biomarkers may be added to a biological sample from a patient suspected to have TBI. In some embodiments, the traumatic brain injury biomarker is selected from the group consisting of: S100B, GFAP, NLF, NFH, γ-enolase (NSE), α-II spectrin, UCH-L1, total tau, and phosphorylated tau. In some embodiments, the traumatic brain injury biomarker is selected from GFAP and UCH-L1.

In some embodiments, the methods described herein (e.g., methods of enrichment) are used to isolate or enrich the presence of one, two, three, four, five, six, seven, eight, or nine of the traumatic brain injury biomarkers selected from the group consisting of: S100B, GFAP, NLF, NFH, γ-enolase (NSE), α-II spectrin, UCH-L1, total tau, and phosphorylated tau.

Alzheimer's Disease. In one embodiment, the biomarker is for Alzheimer's Disease. There are two (2) biomarkers associated with the severity and magnitude of Alzheimer's Disease. In some embodiments, the Alzheimer's Disease biomarker is selected from the group consisting of: amyloid beta, BACE1, and soluble Aβ precursor protein (sAPP). In some embodiments, the Alzheimer's Disease biomarker is selected from the group consisting of: β-amyloid (1-42), phospho-tau (181p), and total-tau. In some embodiments, the methods described herein (e.g., methods of enrichment) are used to isolate or enrich the presence of one, two or three of the Alzheimer's Disease biomarkers selected from the group consisting of: amyloid beta, BACE1, and soluble Aβ precursor protein (sAPP). In some embodiments, the biomarker is amyloid beta, BACE1, or soluble Aβ precursor protein (sAPP). In some embodiments, the biomarker for Alzheimer's Disease is found in a biological sample (e.g., CSF).

Sexually Transmitted Diseases. In one embodiment, the biomarker is for a sexually transmitted disease (STD). There are at least ten (10) biomarkers characteristic of transmission of a STD. In some embodiments, the STD biomarker is a biomarker for Chlamydia, Gonorrhea, Syphilis, Trichomonas, HPV, Herpes 1 and 2, HSV, Hepatitis A, Hepatitis B, Hepatitis C, HIV 1 and 2. In some embodiments, the methods described herein (e.g., methods of enrichment) are used to isolate or enrich the presence of one, two, three, four, five, six, seven, eight, nine, ten, eleven, twelve or thirteen STD biomarkers: Chlamydia, Gonorrhea, Syphilis, Trichomonas, HPV, Herpes 1 and 2, HSV, Hepatitis A, Hepatitis B, Hepatitis C, HIV 1 and 2, and HIV antibodies. In some embodiments, the biomarker is in urine (e.g., Chlamydia, Gonorrhea, Trichomonas). In some embodiments, the biomarker is in blood, serum, or plasma (e.g., Syphilis, HPV, Herpes 1 and 2, HSV, Hepatitis A, Hepatitis B, Hepatitis C, HIV 1 and 2, HIV antibodies).

Bacterial Infection. In one embodiment, the biomarker is for a bacterial infection, e.g., sepsis. The current gold standard test for bacterial infection is blood culture which can take 24-48 hours before a positive result can be reflexed to a confirmatory test such as molecular diagnostics. Described herein are methods to rule-in/rule-out bacterial infection in as little as 30 minutes or less where time is critical to successfully treat patients to prevent or manage sepsis, for example in 60 minutes or less (e.g., 50 minutes, 40 minutes, 30 minutes, 20 minutes or less). There are at least thirty (30) biomarkers characteristic of bacterial infection. In some embodiments, the bacterial biomarker is selected from the group consisting of a biomarker for sepsis-causing species of bacteria (e.g., Enterococcus faecium, Escherichia coli, Klebsiella pneumoniae, Pseudomonas aeruginosa, and Staphylococcus aureus). In some embodiments, the biomarker is a biomarker for Enterococcus faecium, Escherichia coli, Klebsiella pneumoniae, Pseudomonas aeruginosa, and Staphylococcus aureus. In some embodiments, the biomarker is a biomarker for a gram positive or gram negative bacteria. In some embodiments, the biomarker is a biomarker for a yeast pathogen (e.g., a yeast pathogen associated with bloodstream pathogens).

In some embodiments, the gram positive bacteria is: Enterococcus, Listeria monocytogenes, Staphylococcus, Staphylococcus aureus, Streptococcus, Streptococcus agalactiae, Streptococcus pneumoniae, or Streptococcus pyogenes.

In some embodiments, the gram negative bacteria is: Acinetobacter baumannii, Haemophilus influenza, Neisseria meningitides, Pseudomonas aeruginosa, Enterobacteriaceae, Enterobacter cloacae complex, Escherichia coli, Klebsiella oxytoca, Klebsiella pneumoniae, Proteus, or Serratia marcescens.

In some embodiments, the yeast pathogen is: Candida albicans, Candida glabrata, Candida krusei, Candida parapsilosis, Candida tropicalis.

In some embodiments, followed by mass spectrometry method. Method is envisioned where a cleavage agent (e.g., reducing agent (e.g., DTT or TCEP)) is added to a bacteria-particle bound complex to cleave the linker (i.e., linker conjugating particle to surface capture moiety). The resultant bacterial is grown in culture or analyzed by MALDI-TOF mass spectrometry.

Method is envisioned where a cleavage agent (e.g., reducing agent (e.g., DTT or TCEP)) is added to a bacteria-particle bound complex to cleave the linker (i.e., linker conjugating particle to surface capture moiety). The resultant bacterial is grown in culture or analyzed by MALDI-TOF mass spectrometry or analyzed by a molecular diagnostics such as the FilmArray® Blood Culture Identification (BCID) Panel by BioFire Diagnostics.

Pathogen-specific antibodies. In some embodiments the biomarker is an antibody recognizing a pathogen, especially structural or surface exposed antigen of the pathogen. A pathogen-specific antigen is used as a capture moiety on the magnetic particle. Whole blood, a blood fraction, plasma or serum is mixed with the magnetic particle so that the antigen specific antibody can bind to the capture moiety (the pathogen-specific antigen). The particles are magnetically separated from the biological fluid. The magnetic particles are then responded in a release buffer to elute the antibody from the capture moiety. This can be done on an analytic scale and the antibody subjected to mass spectrometry (including, for example, LC-MS to separate multiple species of antibody that may be present), Edman degradation, and other protein chemistry analytic methods to determine the protein sequence of the antibody. The sequence information can be used to construct monoclonal antibodies having the same specificity or specificities (paratope(s)). This can also be done on a preparative scale and the enriched or isolated antigen-specific antibody used in the clinic for therapy or prophylaxis. In some embodiments the antibody recognizing a pathogen-specific antigen recognizes a spike protein of SARS-Cov-2 spike protein, for example the S1 subunit, or a receptor binding domain and/or an N-terminal domain and/or receptor binding domain thereof.

Thyroid Function. TSH concentrations are measured as part of a thyroid function test in patients suspected of having an excess (hyperthyroidism) or deficiency (hypothyroidism) of thyroid hormones. The methods described herein in some embodiments are used to evaluate thyroid function. In some embodiments, the biomarker is an antigen (e.g., TSH). In some embodiments, the capture moiety is an autoantibody (e.g., free autoantibody, complexed autoantibody) with specificity to the antigen (e.g., TSH).

In some embodiments, the interference (affecting measurements of thyroid stimulating hormone, free thyroxine, and free triiodothyronine) is macroTSH, biotin, anti-streptavidin antibodies, anti-ruthenium antibodies, thyroid hormone autoantibodies, or heterophilic antibodies.

Cardiac Function. The methods described herein in some embodiments are used to evaluate cardiac function. An increased level of troponin circulating in blood is a biomarker for heart disorders, e.g., myocardial infarction. Cardiac I and T are specific indicators of heart muscle damage. Subunits of troponin are also markers for cardiac health. Specifically cTnI and cTnT are biomarkers for acute myocardial infarction (AMI) for example type 1 and 2 myocardial infarction, unstable angina, post-surgery myocardium trauma and related diseases. In some embodiments, the biomarker is free cTnI, free cTnT, binary cTnI-TnC, or ternary cTnI-TnC-TnT. In some embodiments, the biomarker is an indicator for heart failure. In some embodiments, the biomarker is an indicator for stroke (e.g., as described in https://www.ahajournals.org/doi/10.1161/STROKEAHA.117.017076 and https://www.360dx.com/business-news/roche-test-helps-differentiate-bleeding-risk-stroke-risk-patients-considering#.W1jz0thKhcA, which are incorporated by reference in their entirety). In some embodiments, the biomarker is an indicator for fibrosis (e.g., as described in http://www.onlinejacc.org/content/65/22/2449, which is incorporated by reference in its entirety). In some embodiments, the biomarker is for diagnosis of acute coronary syndrome (ACS). In some embodiments, the biomarker is for Cardiac Troponin (I, I-C, I-C-T, T) and other cardiac troponin fragments, Natriuretic Peptides (BNP, ANP, CNP), N-terminal fragments (i.e. NT-proBNP, NT-proCNP), glycosylated, non-glycosylated, CRP, Myoglobin, Creatinine kinase (CK), CK-MB, sST2, GDF-15, Galectin-3.

In some embodiments, the accuracy and precision by being able to test large sample volumes (i.e. 1 mL, 10 mL, 100 mL, 1000 mL, etc.) to improve likelihood of detection of very dilute or low concentration biomarker(s), as well as very small sample volumes (i.e. neonates, pediatrics, elderly) which typically are untestable today or require sample dilution before testing which compromises test sensitivity, accuracy and precision. In some embodiments, the biological sample is in a 1 mL, 10 mL, 100 mL, 1000 mL or greater volume. In some embodiments, the biological sample is in a 0.5 mL, 0.25 mL, 0.1 mL, 0.05 mL or lesser volume.

Also provided herein is a method for using particle sample pre-treatment to aid in enrichment of biomarkers prior to a diagnostic test by allowing a wash step or particle isolation followed by selective release or elution of the captured biomarker(s), or selective release or elution of the capture moiety-biomarker complex, from the particles prior to the biomarker characterization step or test method.

The use of a “cleavage reagent or “releasing agent” that will disrupt the bond between the capture moiety on the particle surface and the biomarker, e.g., acidic or basic pH, high molarity salt, sugar, chemical displacer, detergent, surfactant, and/or chelating agent, or combination thereof, without displacing or eluting the capture moiety but only the biomarker. After washing or isolating the particles from the sample matrix with magnet(s), the particles can subsequently be treated with an elution solution containing a releasing agent(s) to selectively release the biomarker and/or labeled detection reagent into solution. The particles can be quickly (less than 2 minutes; ideally less than 30 seconds) isolated to the side(s) and/or bottom of the sample device (vial, test tube, other) to form an essentially particle-free sample supernatant. The particle-free supernatant can be subsequently aspirated without disrupting the pellet comprising particles and dispensed into a separate transfer tube or injected directly onto the analytical system (i.e. LC-MS/MS or MALDI-TOF) for testing of the biomarker. In some embodiments, the supernatant containing the eluted component is transferred into a neutralization buffer to re-establish a less harsh conditions (such as pH) and preserve the biomarker and/or label from degradation or denaturation by the elution solution.

For example, the cleavage reagent or releasing agent described herein disrupt the binding interaction or cleavable bond as described herein between the particles described herein and a capture moiety described herein, e.g., using elution strategies such as pH (e.g. increased pH with a base such as sodium bicarbonate, decreased pH with an acids such as acetic acid, trichloroacetic acid, sulfosalicylic acid, HCl, formic acid, and common pH elution buffers such as 100 mM glycine.HCl, pH 2.5-3.0, 100 mM citric acid, pH 3.0, 50-100 mM triethylamine or triethanolamine, pH 11.5, 150 mM ammonium hydroxide, pH 10.5), a displacer or displacing agent, competitive elution (e.g. >0.1M counter ligand or analog), ionic strength and/or chaotropic effects (e.g. NaCl, KCl, 3.5-4.0M magnesium chloride pH 7.0 in 10 mM Tris, 5M lithium chloride in 10 mM phosphate buffer pH 7.2, 2.5M sodium iodide pH 7.5, 0.2-3.0M sodium thiocyanate), surfactant, detergent, a concentrated inorganic salt, denaturing (e.g. 2-6M guanidine.HCl, 2-8M urea, 1% deoxycholate, 1% SDS), an organic solvent (e.g. alcohol, chloroform, ethanol, methanol, acetonitrile, hexane, DMSO, 10% dioxane, 50% ethylene glycol pH 8-11.5 (also chaotropic)), radiation or heat (increased temperature), conformational change, disulfide bond reducers (2-mercaptoethanol, dithiothreitol, tris(2-carboxylethyl)phosphine), enzyme inactivation, chaotropic agents (Urea, Guanidinium chloride, Lithium perchlorate), mechanical agitation, sonication, and protein digestive enzymes (pepsin, trypsin), and combinations thereof. In some embodiments, the supernatant containing the eluted, component is transferred into a neutralization buffer to re-establish a less harsh conditions (such as pH) and preserve the biomarker and/or label from degradation or denaturation by the elution solution

### Method of Characterization

Described herein are methods for depleting and/or enriching biomarkers for subsequent characterization or diagnostic testing. Characterization of a biomarker described herein (e.g., interference) includes identification and/or quantification of a biomarker described herein (e.g., interference described herein).

Characterization can include detection and/or quantitation of the biomarker, for example, an antigen-specific antibody. By binding an antigen-specific antibody to the particle it can be isolated away from other specificities, and released into a smaller volume than the original sample, concentrating it, if desired. Typical diagnostic assays for specific antibodies detect them without first isolating the antibodies, for example, in the context of whole serum. This makes absolute quantitation difficult; antibody activity is often characterized as a titer, based on how much the serum can be diluted but still retain activity. By capturing the species of interest and separating it from the rest of the immunoglobulins in the serum, a simple protein assay can be used to quantitate how much specific antibody is present. Further, using isotype-specific reagents one can separately quantitate each isotype of interest, in parallel aliquots or, if conjugated to distinct labels, multiplexed in a single aliquot, by comparison to a standard curve generated with known amounts of bead-bound immunoglobulin. The standard curve can be for immunoglobulin generally or for specific isotypes, and the latter can be generated separately or in multiplex fashion. IgM is typical of an early response, whereas IgG and IgA are typical of more mature and more effective immune responses. This easy absolute quantitation makes direct comparison of the amount of antigen-specific antibody in a sera possible from one serum sample to the next. In some embodiments, the antigen-specific antibody recognizes the SARS-CoV-2 S1 subunit, or a receptor binding domain and/or an N-terminal domain and/or receptor binding domain thereof.

### Particles of the Invention

Described herein are particles for the isolation, depletion and/or enrichment of biological samples. In some embodiments, the particles comprise a cleavable bond and a capture moiety (e.g., a particle surface functionalized to present one capture moiety. In some embodiments, the particles comprise a non-cleavable bond and a capture moiety (e.g., a particle surface functionalized to present one capture moiety. In some embodiments, the particles described herein comprise a capture moiety (e.g., a capture moiety with high specificity to a biomarker described herein). In some embodiments, the particles described herein (e.g., the surface of the particles described herein, the particle surface not bound to a capture moiety described herein) are inert (e.g. do not exhibit significant binding to a biomarker described herein). In some embodiments, the particles described herein can be used in the diagnostic tests described herein without further modification to the particle or the diagnostic test. In some embodiments, the particles described herein can be added to and removed from a sample without altering the sample (e.g., without adding or removing an additional biomarker (e.g., an interference).

The particles described herein are sufficiently small with a mean diameter from 0.050 micrometers up to 3.00 micrometers, or preferably from 0.100 micrometers to 1.1 micrometers in diameter, or still more preferably 0.200 micrometers to 0.600 micrometers, or even more preferably from 0.100 micrometers to 0.500 micrometers in diameter.

In some embodiments, the particles described herein (e.g., microparticle, nanoparticle) comprise a core or support, wherein the core or support is a paramagnetic or superparamagnetic material selected from the group consisting of iron oxide, ferromagnetic iron oxide, Fe₂O₃, and Fe₃O₄, maghemite, or combinations thereof.

In some embodiments, the particle surface comprises an organic polymer or copolymer, wherein the organic polymer or copolymer is hydrophobic. In some embodiments, the particle (e.g., nanoparticle, microparticle) surface comprises an organic polymer or copolymer such as a material selected from the group consisting of, but not limited to, ceramic, glass, a polymer, a copolymer, a metal, latex, silica, a colloidal metal such as gold, silver, or alloy, polystyrene, derivatized polystyrene, poly(divinylbenzene), styrene-acylate copolymer, styrene-butadiene copolymer, styrene-divinylbenzene copolymer, poly(styrene-oxyethylene), polymethyl methacrylate, polymethacrylate, polyurethane, polyglutaraldehyde, polyethylene imine, polyvinylpyrrolidone, polyvinyl alcohol, polyacrylic acid, N,N′-methylene bis-acrylamide, polyolefeins, polyethylene, polypropylene, polyvinylchloride, polyacrylonitrile, polysulfone, poly(ether sulfone), pyrolized materials, block copolymers, and copolymers of the foregoing, silicones, or silica, methylol melamine, a biodegradable polymer such as dextran or poly(ethylene glycol)-dextran (PEG-DEX), or combinations thereof.

As used herein, “blocker” refers to a protein, polymer, surfactant, detergent, or combinations thereof. In some embodiments, the binding of a capture moiety on a particle described herein (e.g., nanoparticle, microparticle) is blocked with a blocker such as a protein, polymer, surfactant, detergent, or combinations thereof. The blocker is selected from the group consisting of a protein such as albumin, bovine serum albumin, human serum albumin, ovalbumin, gelatin, casein, acid hydrolyzed casein, gama globulin, purified IgG, animal serum, polyclonal antibody, and monoclonal antibody, a polymer such as polyvinyl alcohol (PVA) and polyvinylpyrrolidone (PVP), a combination of a protein and polymer, a peptide, a PEGylation reagent such as (PEO)n-NHS or (PEO)n-maleimide, a triblock copolymer such as Pluronic F108, F127, and F68, a non-ionic detergent such as Triton X-100, polysorbate 20 (Tween-20), and Tween 80 (non-ionic), a zwitterionic detergent such CHAPS, a ionic detergent such as sodium dodecyl sulfate (SDS), deoxycholate, cholate, and sarkosyl, a surfactant, a sugar such as sucrose, and a commercial blocker such as Heterophilic Blocking Reagent (Scantibodies), MAK33 (Roche Diagnostics), Immunoglobulin Inhibiting Reagent (IIR) (Bioreclamation), Heteroblock (Omega Biologicals), Blockmaster (JSR), TRU Block (Meridian Life Sciences), and StabilCoat® & StabilGuard® (Surmodics). In some embodiments, the blocker is bound to a particle described herein (e.g., covalently bound, non-covalently bound). In some embodiments, the blocker is not bound (e.g., covalently bound, non-covalently bound) to a particle described herein.

Cleavable bond. In an aspect, capture moiety binds to a biomarker by a cleavable bond described herein. The cleavable bond can be through covalent or non-covalent binding. Examples of non-covalent binding include, affinity, ionic, van der Waals (e.g., dipole/dipole or London forces), hydrogen bonding (e.g., between polynucleotide duplexes), and hydrophobic interactions. Where association is non-covalent, the association between the entities is preferably specific. Non-limiting examples of specific non-covalent associations include the binding interaction between biotin and a biotin-binding protein such as avidin, captavidin, SA, neutravidin, a fragment of SA, a fragment of avidin, a fragment of neutravidin, or mixtures thereof; the binding of a biotinylated Fab, a biotinylated immunoglobulin or fragment thereof, a biotinylated small molecule (such as, for example, a hormone ora ligand of a receptor), a biotinylated polynucleotide, a biotinylated macromolecule (e.g., a protein ora natural or synthetic polymer) to a biotin-binding protein such as avidin, SA, neutravidin, a fragment of SA, a fragment of avidin, a fragment of neutravidin, or mixtures thereof; the binding of a substrate to its enzyme; the binding of a glycoprotein to a lectin specific for the glycoprotein; the binding of a ligand to a receptor specific for the ligand; the binding of an antibody to an antigen against which the antibody is raised; and duplex formation between a polynucleotide and a complementary or substantially complementary polynucleotide; etc.

A cleavable bond, such as a disulfide bond (R-S-S-R) is used to immobilize or bind the capture moiety (i.e. antibody or antibody fragment such as SH-Fab) to the particle. After washing or isolating the particles from the sample matrix, the particles can subsequently be treated with a solution containing a reducing agent such as TCEP or DTT to cleave the disulfide bond and release the capture moiety-biomarker complex into a solution. The particles can be quickly (less than 2 minutes; ideally less than 30 seconds) isolated to the side(s) and/or bottom of the sample device (vial, test tube, other) to form an essentially particle-free sample supernatant. The particle-free supernatant can be subsequently aspirated without disrupting the pellet comprising particles and dispensed into a separate transfer tube or injected directly onto the analytical system (i.e. LC-MS/MS or MALDI-TOF or molecular diagnostics such as the FilmArray Blood Culture Identification Panel) for testing of the capture moiety-biomarker complex.

In some embodiments, the cleavable bond is a disulfide bond (R-S-S-R).

In some embodiments, the cleavable bond is a non-covalent bond between streptavidin or captavidin, avidin, and biotin.

Capture Moieties. Provided herein are particles comprising one capture moiety that bind an interference as described herein, or a biomarker as described here. As referred to herein, “capture moiety” is selected from the group consisting of an antibody, a binding fragment of an antibody, a IgG, a IgM, a IgA, IgE, IgD a receptor, a ligand of a receptor, a hormone, a receptor of a hormone, an enzyme, a substrate of an enzyme, a single stranded oligonucleotide, a single stranded polynucleotide, a double stranded oligonucleotide, a double stranded polynucleotide, an antigen, a peptide, a polymer, an aptamer, and a protein.

In some embodiments, the capture moiety is a protein. A protein can be, for example, a monomer, a dimer, a multimer, or a fusion protein. In specific embodiments, the protein comprises at least one of an albumin such as, for example, antibody, a fragment of an antibody, BSA, ovalbumin, a fragment of BSA, a fragment of ovalbumin, mouse IgG, polymerized mouse IgG, antibody fragments (Fc, Fab, F(ab′)2) and different subclasses (IgG1, IgG2a, IgG2b, IgG3, IgE, IgD) of mouse IgG to target HAMA and RF interference mechanisms, purified animal polyclonal antibodies (i.e. bovine, goat, mouse, rabbit, sheep) to target HAAA interference, streptavidin, ALP, HRP, BSA (conjugated to isoluminol, ruthenium, acridinium) to target MASI interference or mixtures thereof. In some embodiments the capture moiety is a structural or surface exposed antigen of the pathogen, for example a bacterium or virus. In some embodiments, the capture moiety is a viral structural protein, such as the spike protein of Severe acute respiratory syndrome coronavirus 2 (SARS-CoV-2). In some embodiments, the spike protein is the S1 subunit, or a receptor binding domain and/or an N-terminal domain thereof.

In some embodiments, the capture moiety is a human anti-animal antibody (e.g., mouse IgG, sheep IgG, goat IgG, rabbit IgG, cow IgG, pig IgG, horse IgG). In some embodiments, the capture moiety is a heterophilic antibody (e.g., FR (Fc-specific), Fab, F(ab)′2, polymerized IgG (type 1, 2a, 2b IgG and IgG fragments, serum components). In some embodiments, the capture moiety is an assay specific binder (e.g., biotin, fluorescein, anti-fluorescein poly/Mab, anti-biotin poly/Mab, streptavidin, neutravidin). In some embodiments, the capture moiety is an assay specific signal molecule (e.g., HRP, ALP, acridinium ester, isoluminol/luminol, ruthenium, ABEI/cyclic ABEI). In some embodiments, the capture moiety is an assay specific blocker (e.g., BSA, fish skin gelatin, casein, ovalbumin, PVP, PVA). In some embodiments, the capture moiety is an assay specific conjugate linker (e.g., LC, LC-LC, PEO4, PEO16). In some embodiments, the capture moiety is an antigen autoantibody (e.g., free T3, free T4). In some embodiments, the capture moiety is a protein autoantibody (e.g., MTSH, TnI, TnT, non-cardiac TnT (skeletal muscle disease)). In some embodiments, the capture moiety is a chemiluminescent substrate (e.g., luminol, isoluminol, isoluminol derivatives, ABEI, ABEI derivatives, ruthenium, acridinium ester) or fluorescent label (e.g., fluorescein or other fluorophores and dyes). In some embodiments, the capture moiety is streptavidin, neutravidin, avidin, polyA, polyDT, aptamers, antibodies, Fab, F(ab′)2, antibody fragments, recombinant proteins, enzymes, proteins, biomolecules, polymers, or molecularly imprinted polymers. In some embodiments, the capture moiety is biotin, fluorescein, Po1yDT, PolyA, antigen, etc.

In some embodiments, the capture moiety binds biotin (e.g., avidin, streptavidin, neutravidin, CaptAvidin, anti-biotin antibody, antibody fragment, aptimer, molecularly imprinted polymer, etc.)

Some embodiments provide a binding surface with two or more different capture moieties.

Generation of Capture Moieties. In an aspect, provided is a method for making a capture moiety, the method comprising the production or generation of complex-specific or conformation-specific antibodies to free autoantibodies or autoantibody complexes. Free autoantibodies are autoantibodies that are not already complexed to their antigen target. Complexed autoantibodies are autoantibodies that have formed a complex with their antigen target.

In an aspect, provided is a method for making a capture moiety, the method comprising the production or generation of complex-specific or conformation-specific antibodies to autoantibody complexes like MTSH. In some embodiments, the autoantibody is triiodothyronine (T3) or thyroxine (T4). In some embodiments, the autoantibody complex is MTSH. For example, complex-specific or conformation-specific antibodies can be raised to autoantibody complexes like MTSH, which can be purified from human serum and used as the capture moiety. In this way the antibodies generated would only have specificity to hlgG or hlgM complexes with TSH. MTSH can be purified based on techniques and published methods or by someone skilled in the art of protein biochemistry and purification. In some embodiments, patients with autoimmune disease who have the greatest likelihood of autoantibody assay interference are used to produce or generate autoantibodies. For example, see the HyTest SES assay for BNP, WO2014114780, WO2016113719 and WO2016113720, the references of which are cited in their entirety.

Thyroid-specific Autoantibodies. For example, in an embodiment, the autoantibody is an anti-thyroid autoantibody (e.g., anti-thyroid peroxidase antibody, thyrotropin receptor antibodies, thyroglobulin antibodies). Anti-thyroid autoantibodies are autoantibodies targeted against one or more components on the thyroid.

In some embodiments, the autoantibody is a free autoantibody (e.g., thyrotropin (TSH).

In some embodiments, the autoantibody is a complexed autoantibody (e.g., MTSH). In some embodiments, the capture moieties described herein are antibodies generated with specificity to complexed autoantibodies or with confirmation specificity to the hlgG and/or hlgM already bound to its antigen target such as MTSH.

Itemized below is a nonlimiting list of substances that may function as one, or alternatively as the other, member of a binding pair consisting of analyte binder (capture moiety) and analyte, depending on the application for which an affinity assay is to be designed. Such substances can be used, for example, as capture moieties (analyte binders) or can be used to generate capture moieties (e.g., by employing them as haptens/antigens to generate specific antibodies) that can be used with the various embodiments. Affinity assays, including immunoassays, can be designed in accordance with the various embodiments to detect the presence and/or level of such substances where they are analytes in a sample. In a specific embodiment, the analyte-binding capture moieties can be used to detect these substances as analytes in a sample. Alternatively, the herein disclosed substances can be associated with the solid phase support surface in accordance with the various embodiments, and used to capture molecules that interact with them (such as, for example, antibodies or fragments thereof specific for the listed substances, binding proteins, or enzymes).

A nonlimiting list of substances that may function as one, or alternatively as the other, member of a binding pair consisting of analyte binder (capture moiety) and analyte includes: inducible nitric oxide synthase (iNOS), CA19-9, IL-Iα, IL-1β, IL-2, IL-3, IL-4, IL-t, IL-5, IL-7, IL-10, IL-12, IL-13, sIL-2R, sIL-4R, sIL-6R, SIV core antigen, IL-1RA, TNF-α, IFN-gamma, GM-CSF; isoforms of PSA (prostate-specific antigen) such as PSA, pPSA, BPSA, in PSA, non α₁-antichymotrypsin-complexed PSA, α₁-antichymotrypsin-complexed PSA, prostate kallikreins such as hK2, hK4, and hK15, ek-rhK2, Ala-rhK2, TWT-rhK2, Xa-rhK2, HWT-rhK2, and other kallikreins; HIV-1 p24; ferritin, L ferritin, troponin I, BNP, leptin, digoxin, myoglobin, B-type natriuretic peptide or brain natriuretic peptide (BNP), NT-proBNP, CNP, NT- proCNP(1-50), NT-CNP-53(51-81), CNP-22(82-103), CNP-53(51-103), atrial natriuretic peptide (ANP); human growth hormone, bone alkaline phosphatase, human follicle stimulating hormone, human leutinizing hormone, prolactin; human chorionic gonadotrophin (e.g., CGα, CGβ), soluble ST2, thyroglobulin; anti-thyroglobulin; IgE, IgG, IgG1, IgG2, IgG3, IgG4, B. anthracis protective antigen, B. anthracis lethal factor, B. anthracis spore antigen, F. tularensis LPS, S. aureas enterotoxin B, Y. pestis capsular F1 antigen, insulin, alpha fetoprotein (e.g., AFP 300), carcinoembryonic antigen (CEA), CA 15.3 antigen, CA 19.9 antigen, CA 125 antigen, HAV Ab, HAV Igm, HBc Ab, HBc Igm, HIV1/2, HBsAg, HBsAb, HCV Ab, anti-p53, histamine; neopterin; s- VCAM-1, serotonin, sFas, sFas ligand, sGM-CSFR, s1CAM-1, thymidine kinase, IgE, EPO, intrinsic factor Ab, haptoglobulin, anti-cardiolipin, anti-dsDNA, anti-Ro, Ro, anti-La, anti-SM, SM, anti-nRNP, antihistone, anti-Scl-70, Scl-70, anti-nuclear antibodies, anti-centromere antibodies, SS-A, SS-B, Sm, U1-RNP, Jo-1, CK, CK-MB, CRP, ischemia modified albumin, HDL, LDL, oxLDL, VLDL, troponin T, troponin I, troponin C, microalbumin, amylase, ALP, ALT, AST, GGT, IgA, IgG, prealbumin, anti-streptolysin, chlamydia, CMV IgG, toxo IgG, toxo IgM, apolipoprotein A, apolipoprotein B, C3, C4, properdin factor B, albumin, α₁-acid glycoprotein, α₁-antitrypsin, α₁-microglobulin, α₂-macroglobulin, anti-streptolysin O, antithrombin-III, apolipoprotein AI, apolipoprotein B, β₂-microglobulin, ceruloplasmin, complement C3, complement C4, C-reactive protein, DNase B, ferritin, free kappa light chain, free lambda light chain, haptoglobin, immunoglobulin A, immunoglobulin A (CSF), immunoglobulin E, immunoglobulin G, immunoglobulin G (CSF), immunoglobulin G (urine), immunoglobulin G subclasses, immunoglobulin M, immunoglobulin M (CSF), kappa light chain, lambda light chain, lipoprotein (a), microalbumin, prealbumin, properdin factor B, rheumatoid factor, ferritin, transferrin, transferrin (urine), rubella IgG, thyroglobulin antibody, toxoplasma IgM, toxoplasma IgG, IGF-I, IGF-binding protein (IGFBP)-3, hepsin, pim-1 kinase, E-cadherein, EZH2, and a-methylacyl-CoA racemase, TGF-beta, IL6SR, GAD, IA-2, CD-64, neutrophils CD-64, CD-20, CD-33, CD-52, isoforms of cytochrome P450, s-VCAM-1, sFas, sICAM, hepatitis B surface antigen, thromboplastin, HIV p24, HIV gp41/120, HCV C22, HCV C33, hemoglobin A1c, and GAD65, IA2, vitamin D, 25-OH vitamin D, 1,25(OH)2 vitamin D, 24,25(OH)2 vitamin D, 25,26(OH)2 vitamin D, 3-epimer of vitamin D, FGF-23, sclerostin, procalcitonin, calcitonin, c. dificille toxin A&B, h. pylori, HSV-1, HSV2.

Suitable substances that may function as one, or alternatively as the other, member of a binding pair consisting of analyte binder (capture moiety) and analyte, depending on the application for which an affinity assay is to be designed, and that can be used with the presently disclosed embodiments also include moieties, such as for example antibodies or fragments thereof, specific for any of the WHO International Biological Reference Preparations held and, characterized, and/or distributed by the WHO International Laboratories for Biological Standards (available at http:/www.who.int/bloodproducts/re_materials, updated as of Jun. 30, 2005, which lists substances that are well known in the art; the list is herein incorporated by reference).

A partial list of such suitable international reference standards, identified by WHO code in parentheses following the substance, includes: human recombinant thromboplastin (rTF/95), rabbit thromboplastin (RBT/90), thyroid-stimulating antibody (90/672), recombinant human tissue plasminogen activator (98/714), high molecular weight urokinase (87/594), prostate specific antigen (96/668), prostate specific antigen 90:10 (96/700); human plasma protein C (86/622), human plasma protein S (93/590), rheumatoid arthritis serum (W1066), serum amyloid A protein (92/680), streptokinase (00/464), human thrombin (01/580), bovine combined thromboplastin (OBT/79), anti-D positive control intravenous immunoglobulin (02/228), islet cell antibodies (97/550), lipoprotein a (IFCC SRM 2B), human parvovirus B19 DNA (99/800), human plasmin (97/536), human plasminogen-activator inhibitor 1 (92/654), platelet factor 4 (83/505), prekallikrein activator (82/530), human brain CJD control and human brain sporadic CJD preparation 1 and human brain sporadic CJD preparation 2 and human brain variant CJD (none; each cited in WHO TRS ECBS Report No. 926, 53.sup.rd Report, brain homogenate), human serum complement components C1q, C4, C5, factor B, and whole functional complement CH50 (W1032), human serum immunoglobulin E (75/502), human serum immunoglobulins G, A, and M (67/86), human serum proteins albumin, alpha- 1-antitrypsin, alpha-2-macroglobulin, ceruloplasmin, complement C3, transferrin (W1031), anti-D negative control intravenous immunoglobulin (02/226), hepatitis A RNA (00/560), hepatitis B surface antigen subtype adw2 genotype A (03/262 and 00/588), hepatitis B viral DNA (97/746), hepatitis C viral RNA (96/798), HIV-1 p24 antigen (90/636), HIV-1 RNA (97/656), HIV-1 RNA genotypes (set of 10 I01/466), human fibrinogen concentrate (98/614), human plasma fibrinogen (98/612), raised A2 hemoglobin (89/666), raised F hemoglobin (85/616), hemoglobincyanide (98/708), low molecular weight heparin (85/600 and 90/686), unfractionated heparin (97/578), blood coagulation factor VIII and von Willebrand factor (02/150), human blood coagulation factor VIII concentrate (99/678), human blood coagulation factor XIII plasma (02/206), human blood coagulation factors II, VII, IX, X (99/826), human blood coagulation factors II and X concentrate (98/590), human carcinoembryonic antigen (73/601), human C-reactive protein (85/506), recombinant human ferritin (94/572), apolipoprotein B (SP3-07), beta-2-microglobulin (B2M), human beta- thromboglobulin (83/501), human blood coagulation factor IX concentrate (96/854), human blood coagulation factor IXa concentrate (97/562), human blood coagulation factor V Leiden, human gDNA samples FV wild type, FVL homozygote, FVL heterozygote (03/254, 03/260, 03/248), human blood coagulation factor VII concentrate (97/592), human blood coagulation factor VIIa concentrate (89/688), human anti-syphilitic serum (HS), human anti-tetanus immunoglobulin (TE-3), human antithrombin concentrate (96/520), human plasma antithrombin (93/768), human anti-thyroglobulin serum (65/93), anti-toxoplasma serum (TOXM), human anti-toxoplasma serum (IgG) (01/600), human anti-varicella zoster immunoglobulin (W1044), apolipoprotein A-1 (SP1-01), human anti-interferon beta serum (G038-501-572), human anti-measles serum (66/202), anti-nuclear ribonucleoprotein serum (W1063), anti-nuclear-factor (homogeneous) serum (66/233), anti-parvovirus B19 (IgG) serum (91/602), anti-poliovirus serum Types 1,2,3 (66/202), human anti-rabies immunoglobulin (RAI), human anti-rubella immunoglobulin (RUBI-1-94), anti-smooth muscle serum (W1062), human anti-double-stranded DNA serum (Wo/80), human anti-E complete blood-typing serum (W1005), human anti-echinococcus serum (ECHS), human anti-hepatitis A immunoglobulin (97/646), human anti-hepatitis B immunoglobulin (W1042), human anti-hepatitis E serum (95/584), anti-human platelet antigen-1a (93/710), anti-human platelet antigen-5b (99/666), human anti-interferon alpha serum (B037-501-572), human alphafetoprotein (AFP), ancrod (74/581), human anti-A blood typing serum (W1001), human anti-B blood typing serum (W1002), human anti-C complete blood typing serum (W1004), anti-D (anti-Rh0) complete blood-typing reagent (99/836), human anti-D (anti-Rh0) incomplete blood-typing serum (W1006), and human anti-D immunoglobulin (01/572).

Other examples of suitable substances that may function as one, or alternatively as the other, member of a binding pair consisting of analyte binder (capture moiety) and analyte, depending on the application for which an affinity assay is to be designed include compounds that can be used as haptens to generate antibodies capable of recognizing the compounds, and include but are not limited to, any salts, esters, or ethers, of the following: hormones, including but not limited to progesterone, estrogen, and testosterone, progestins, corticosteroids, and dehydroepiandrosterone, and any non-protein/non-polypeptide antigens that are listed as international reference standards by the WHO. A partial list of such suitable international reference standards, identified by WHO code in parentheses following the substance, includes vitamin B12 (WHO 81.563), folate (WHO 95/528), homocystein, transcobalamins, T4/T3, and other substances disclosed in the WHO catalog of International Biological Reference Preparations (available at the WHO website, for example at page http://www.who.int/bloodproducts/ref_materials/, updated Jun. 30, 2005), which is incorporated herein by reference. The methods and compositions described herein can comprise an aforementioned WHO reference standards or a mixture containing a reference standard.

Other examples of substances that may function as one, or alternatively as the other, member of a binding pair consisting of analyte binder (capture moiety) and analyte, depending on the application for which an affinity assay is to be designed include drugs of abuse. Drugs of abuse include, for example, the following list of drugs and their metabolites (e.g., metabolites present in blood, in urine, and other biological materials), as well any salts, esters, or ethers, thereof: heroin, morphine, hydromorphone, codeine, oxycodone, hydrocodone, fentanyl, demerol, methadone, darvon, stadol, talwin, paregoric, buprenex; stimulants such as, for example, amphetamines, methamphetamine; methylamphetamine, ethylamphetamine, methylphenidate, ephedrine, pseudoephedrine, ephedra, ma huang, methylenedioxyamphetamine (MDS), phentermine, phenylpropanolamine; amiphenazole, bemigride, benzphetamine, bromatan, chlorphentermine, cropropamide, crothetamide, diethylpropion, dimethylamphetamine, doxapram, ethamivan, fencamfamine, meclofenoxate, methylphenidate, nikethamide, pemoline, pentetrazol, phendimetrazine, phenmetrazine, phentermine, phenylpropanolamine, picrotoxine, pipradol, prolintane, strychnine, synephrine, phencyclidine and analogs such as angel dust, PCP, ketamine; depressants such as, for example, barbiturates, gluthethimide, methaqualone, and meprobamate, methohexital, thiamyl, thiopental, amobarbital, pentobarbital, secobarbital, butalbital, butabarbital, talbutal, and aprobarbital, phenobarbital, mephobarbital; benzodiazapenes such as, for example, estazolam, flurazepam, temazepam, triazolam, midazolam, alprazolam, chlordiazepoxide, clorazepate, diazepam, halazepam, lorazepam, oxazepam, prazepam, quazepam, clonazepam, flunitrazepam, GBH drugs such as gamma hydroxyl butyric acid and gamma butyrolactone; glutethimide, methaqualone, meprobamate, carisoprodol, zolpidem, zaleplon; cannabinoid drugs such as tetrahydracannabinol and analogs; cocaine, 3-4 methylenedioxymethamphetamine (MDMA); hallucinogens such as, for example, mescaline and LSD.

### EXAMPLES

### Example 1: Biotin Interference Depletion After High Dose Biotin Ingestion

Endogenous (non-spiked) biotin samples were serially collected. Baseline serum samples were obtained from 5 apparently healthy adult volunteers (4 male, 1 female) by antecubital venous blood draw in BD brand Vacutainer™ 10 mL red top tubes. Each volunteer subsequently ingested a 20 mg dose of biotin (4×5 mg, Finest Nutrition Biotin 5000 mcg Strawberry, Quick Dissolve, Item # 938508, distributed by Walgreens). Serum samples were obtained 1, 3, 6, 8, and 24 hours after biotin ingestion. Blood was allowed to clot for 1 hour at RT and centrifuged at 2,000 rpm for 15 min in a Beckman Allegra 6R tabletop centrifuge. For each time point, serum samples from each volunteer was pooled, mixed for 15 min at RT, aliquoted into 1.2 mL aliquots in 2 mL cryo-vials, and frozen at −80° C.

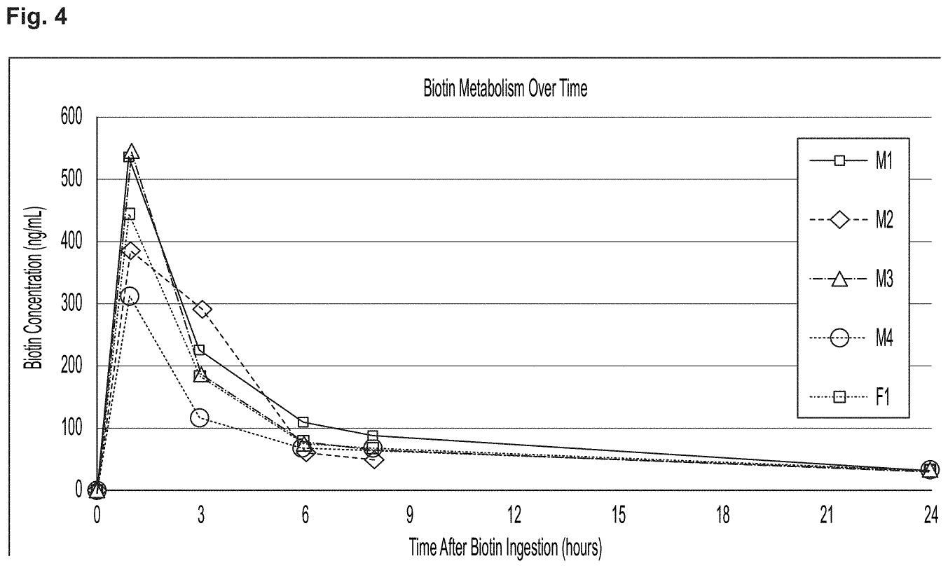

Biotin metabolism was determined by measuring biotin levels in serially collected samples using a free biotin ELISA. The biotin serum samples were tested by the Immundiagnostik IDK® Biotin ELISA kit (Part No. K8141, Lot No. 180906, measuring range of 48.1-1100 pg/mL). Samples above the kit's measuring range were diluted with the kit's sample dilution buffer. Samples collected 1, 3, 6, and 8 hours after biotin ingestion were posited to be in the 50,000 to 500,000 pg/mL range and were diluted 1:1000. Samples collected 24 hours after biotin ingestion were posited to be near or less than 20,000 pg/mL and diluted 1:20. Samples were tested according to the ELISA kit protocol and biotin levels were still significantly elevated 8 hours (60-107 ng/mL; n=5) and 24 hours (24-32 ng/mL; n=4) after 20 mg biotin ingestion (FIG. 4 and Table 1).

TABLE 1

| | Biotin Concentration (ng/mL) | | | | | |
|---|---|---|---|---|---|---|
| Volunteer | 0-hr | 1-hr | 3-hr | 6-hr | 8-hr | 24-hr |
| M1 | 1.1 | 531 | 221 | 107 | 85 | 29 |
| M2 | 0.1 | 385 | 289 | 60 | 48 | Not |
| | | | | | | collected |
| M3 | 0.3 | 546 | 184 | 78 | Not | 26 |
| | | | | | collected | |
| M4 | 0.4 | 308 | 113 | 65 | 64 | 32 |
| F1 | 0.4 | 440 | 183 | 76 | 65 | 24 |

High levels of endogenous biotin (370 or 550 ng/mL) were depleted from the serially collected serum samples using superparamagnetic nanoparticles coated with Streptavidin (VERAPREP Biotin reagent) by adding 200 μL serum to a 1.5 mL microcentrifuge tube, adding 20 μL VERAPREP Biotin reagent, gently mixing/rocking the sample for 10 minutes, magnetically separating VERAPREP Biotin reagent for 10 minutes using a Dexter LifeSep® 1.5S, carefully aspirating the serum to avoid disrupting the magnetic particles, and by testing the serum sample using a free biotin ELISA.

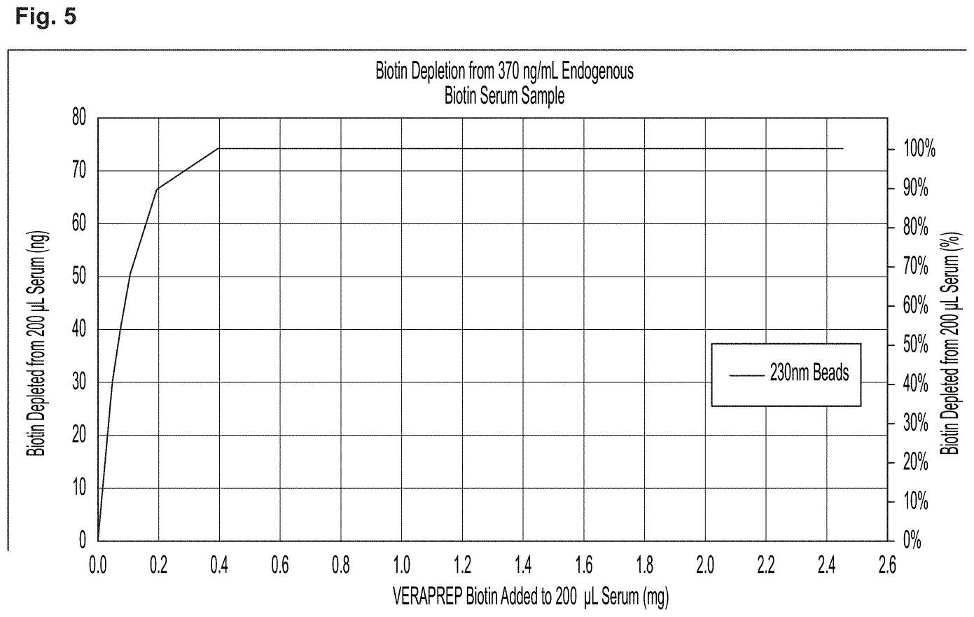

In the first study, increasing amounts (mg) of VERAPREP Biotin reagent was added to different aliquots of the same high biotin (370 ng/mL) endogenous serum sample to determine how much reagent was required to deplete 100% of the free biotin in the sample. 20 μL VERAPREP Biotin reagent (230 nm diameter, 32 μg Streptavidin per mg beads) was added to each 200 μL aliquot of the serum sample collected 1 hour after 20 mg biotin ingestion, mixed by gentle inversion for 10 min at RT, and magnetically separated for 10 min using a Dexter LifeSep 1.5S magnet. 175 μL serum supernatant was carefully aspirated and measured by the Immundiagnostik IDK® Biotin ELISA kit (Part No. K8141, Lot No. 180906) and samples above the kit's measuring range were diluted with the kit's sample dilution buffer. The 230 nm VERAPREP Biotin reagent successfully depleted 100% free biotin using a simple 20 min process, 200 μL sample, and only 0.39 mg reagent (FIG. 5)

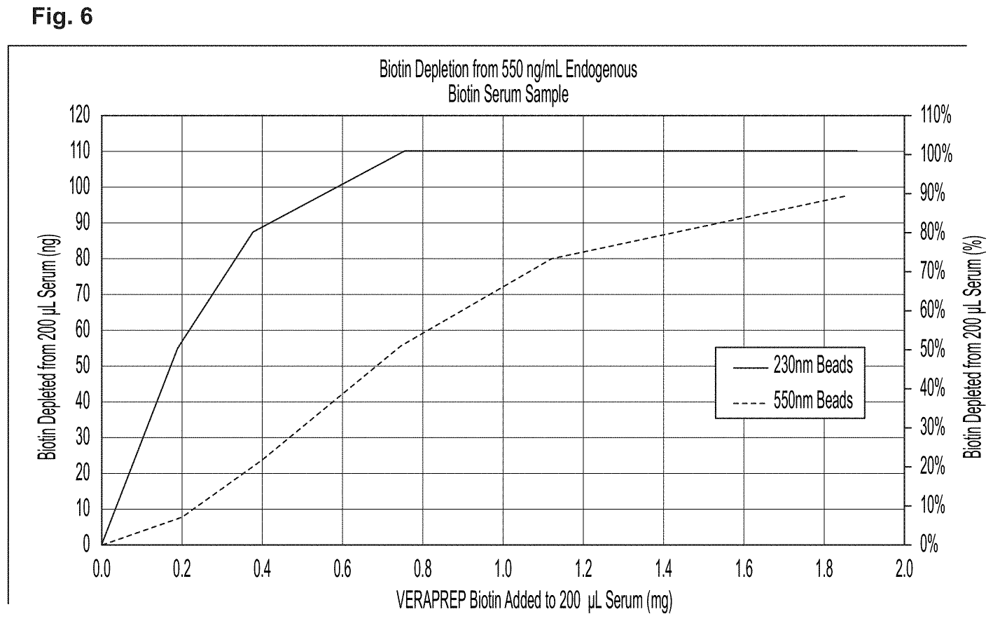

In the second study, increasing amounts (mg) of two different VERAPREP Biotin reagents was added to different aliquots of the same high biotin (550 ng/mL) serum sample to determine how much of each reagent was required to deplete 100% of the free biotin in the sample. 20 μL of 230 nm VERAPREP Biotin reagent (32 μg Streptavidin per mg beads), or 20 μL of 550 nm VERAPREP Biotin reagent (4 μg Streptavidin per mg beads), was added to each 200 μL aliquot of serum collected 1 hour after 50 mg biotin ingestion, mixed by gentle inversion for 10 min at RT, and magnetically separated for 10 min using a Dexter LifeSep 1.5S magnet. 175 μL serum supernatant was carefully aspirated and measured by the Immundiagnostik IDK® Biotin ELISA kit (Part No. K8141, Lot No. 180906) and samples above the kit's measuring range were diluted with the kit's sample dilution buffer. The 230 nm VERAPREP Biotin reagent successfully depleted 100% free biotin using a simple 20 min process, 200 μL sample, and only 0.75 mg reagent, while the 550 nm VERAPREP Biotin reagent only depleted 89% free biotin with 1.86 mg reagent. These results demonstrate that the binding capacity and binding efficiency of VERAPREP Biotin reagent is improved with a smaller bead diameter and increased surface area per unit mass, by increasing the amount of Streptavidin and biotin binding capacity per mg beads, and/or by adding an increased concentration or amount (mg) of the VERAPREP Biotin reagent (FIG. 6).

### Example 2. Biotin Interference Depletion Using an Optimized Sample Pre-Treatment Reagent to Bind and Deplete High Concentrations of Free Biotin in Serum Samples

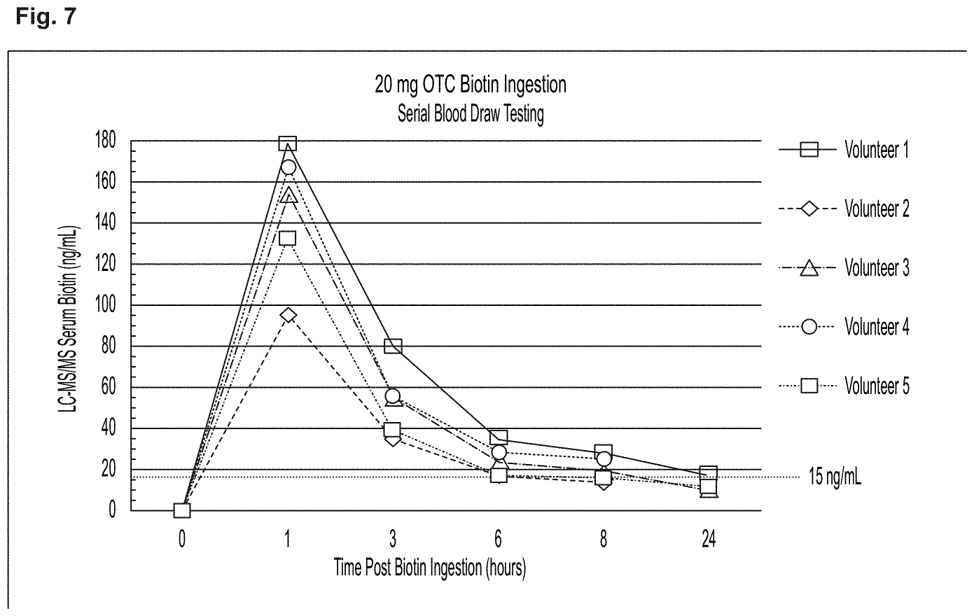

Six volunteers (five apparently healthy adults ages 25 to 46, one Diabetic Type 2 age 65) had fasting serum samples collected at baseline by antecubital venous blood draw in BD brand Vacutainer™ 10 mL red top tubes. Each volunteer subsequently ingested a 20 mg, 100 mg or 200 mg dose of over the counter (OTC) biotin. For the 20 mg dose, serum samples were obtained 1, 3, 6, 8, and 24 hours after biotin ingestion. For the 100 and 200 mg doses, serum samples were collected at 1, 6 and 24 hours after biotin ingestion. Blood was allowed to clot for 1 hour at RT and centrifuged at 2,000 rpm for 15 min in a Beckman Allegra 6R tabletop centrifuge. For each time point and biotin dose, serum samples from each volunteer was pooled, mixed for 15 min at RT, aliquoted into 1.2 mL aliquots in 2 mL cryo-vials, and frozen at −80° C. All samples were sent out for LC-MS/MS biotin measurements at the University of Washington, Department of Laboratory Medicine, 1959 NE Pacific Street, Seattle, Wash. 98195. For the 20 mg dose, biotin levels were highest at 1 hour [96-179 ng/mL], at 6 hours all 5 volunteers still had serum biotin levels >15 ng/mL [17-35], at 8 hours, 4 out of 5 volunteers had serum biotin levels >15 ng/mL [16-28], and at 24 hours Volunteer 1, a known diabetic Type 2, had a biotin level >15 ng/mL [18] (FIG. 7).

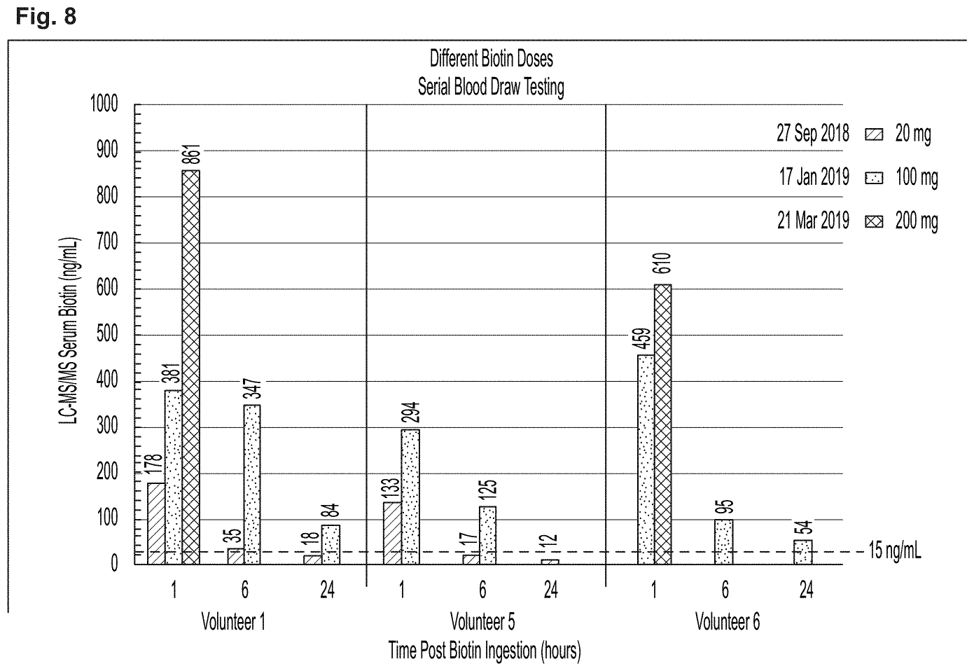

For the 100 and 200 mg doses biotin levels were highest at 1 hour at 294-459 ng/mL for the 100 mg dose, and 610-861 ng/mL for the 200 mg dose. At 6 hours, volunteers who ingested 20 mg or 100 mg biotin had serum biotin levels >15 ng/mL at 17-35 ng/mL for the 20 mg dose, and 95-347 ng/mL for the 200 mg dose. At 24 hours, the 2 volunteers who ingested 100 mg biotin had biotin levels >15 ng/mL with Volunteer 1, known diabetic, at 84 ng/mL, and Volunteer 6 at 54 ng/mL (FIG. 8).

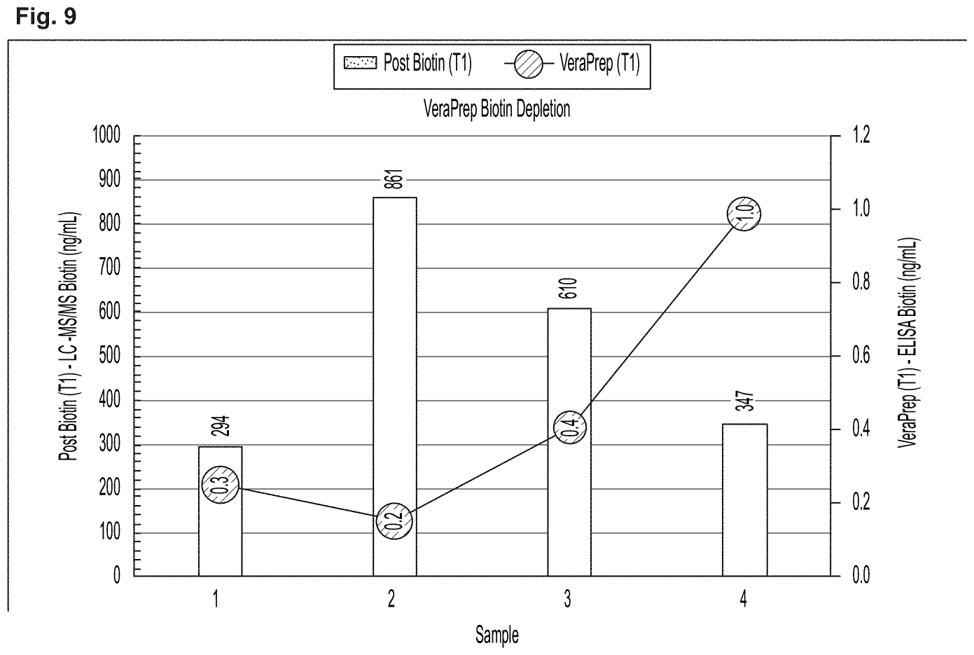

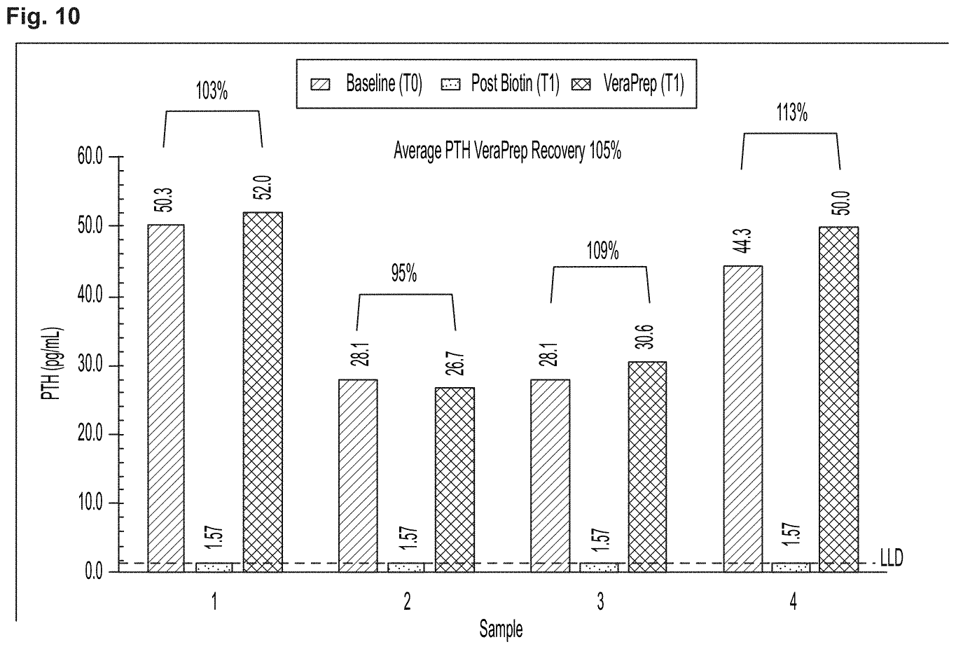

Four samples were selected with high endogenous biotin levels [294-861 ng/mL] measured by LC-MS/MS (FIG. 9). The baseline serum samples were tested by the PTH Intact ELISA (DRG PTH Intact ELISA, Part No. EIA-3645) and PTH values ranged from 28.1 to 50.3 pg/mL. The 1 hour post-biotin ingestion samples all had PTH results were <1.57 pg/mL, or below the lower limit of detection (LLD)(FIG. 10).

All 4 samples were pre-treated with optimized 550 nm superparamagnetic nanoparticles coated with Streptavidin (VeraPrep Biotin), where Samples 1 and 4 had biotin levels <500 ng/mL by LC-MS/MS and were pre-treated with 0.5 mg reagent, and Samples 2 and 3 had biotin levels >500 ng/mL by LC-MS/MS and were pre-treated with 1.5 mg reagent using the following protocol:
1. Remove the VeraPrep Biotin reagent vial from storage and vortex for a minimum of 10 seconds at medium speed to mix well and resuspend the reagent.
2. Insert the reagent vial in the foam vial holder.
3. Insert an empty Micro tube 2ml (SARSEDT Order Number 72.694) into the LifeSep® 1.5S magnet until the collar of the tube contacts the magnet frame.
4. Dispense 200 μL (0.5 mg) or 600 μL (1.5 mg) of the well-mixed reagent into the empty tube to separate the reagent on the magnet for >30 seconds to form a reagent pellet.
5. Carefully aspirate and discard all of the storage buffer supernatant (˜200 μL or ˜600 μL) without disturbing the reagent pellet.
6. Dispense 400 μL of well-mixed serum or plasma sample into the tube containing the reagent pellet.
7. Tighten the screw the cap on the tube, remove the tube from the magnet, and vortex for a minimum of 10 seconds at medium speed to mix well and resuspend the reagent in the sample.
8. Place the tube onto a laboratory mixer at medium speed and incubate at room temperature for 10 minutes.
9. Loosen the screw cap and insert the tube into the magnet until the collar of the tube contacts the magnet frame.
10. Magnetically separate the reagent for >4 minutes to form a reagent pellet.
11. Carefully aspirate the sample supernatant without disturbing the reagent pellet and dispense the sample into a transfer tube for testing. Note: All of the sample supernatant (˜400 μL) can be aspirated if this step is performed carefully. If any of the reagent is accidentally aspirated then simply return the sample/reagent mixture to the tube and return to step 10.
12. The sample is now ready for testing.

To verify biotin interference removal, VeraPrep Biotin pre-treated samples were tested using the Immundiagnostik IDK® Biotin ELISA kit (Part No. K8141, measuring range of 48.1-1,100 ng/L). Biotin concentrations ranged from 0.2 to 1.0 ng/mL, or within normal plasma levels (200-1,200 ng/L) (FIG. 9). Immediately after VeraPrep Biotin pre-treatment of Samples 1-4, PTH values were measured using the PTH Intact ELISA and PTH values ranged from 26.7 to 52.0 pg/mL (FIG. 10).

The 1 hour post-biotin ingestion samples had high levels of biotin interference per LC-MS/MS (294 to 861 ng/mL) and undetectable PTH values by the PTH Intact ELISA (<1.57 pg/mL), while the 1 hour post-biotin ingestion samples pre-treated with VeraPrep Biotin had physiologically normal biotin values per the Biotin ELISA kit (<1.1 ng/mL) and normal PTH values by the PTH Intact ELISA (26.7 to 52.0 pg/mL)(FIGS. 9 and 10). When comparing VeraPrep Biotin sample pre-treatment PTH values to the baseline PTH values the results recovered from 95 to 113% (mean recovery of 105%)(FIG. 10). A significant difference in test results after VeraPrep Biotin sample pre-treatment, or a significant increase in PTH values in this PTH Intact ELISA sandwich immunoassay, confirms biotin interference was clinically significant in all 4 samples tested.

### Example 3: Low Abundance Biomarker Enrichment

Very low levels of a biomarker in 40 mL PBS, either 0.0195 μlU TSH/mL or 0.497 pg PTH/mL) were enriched using 550 nm superparamagnetic nanoparticles coated with Streptavidin and subsequently coated with biotinylated anti-TSH antibody (VERAPREP Concentrate TSH reagent) or biotinylated anti-PTH monoclonal antibody (VERAPREP Concentrate PTH reagent).

In the first study, VERAPREP Concentrate TSH reagent was prepared by coating 550 nm VERAPREP Biotin with biotinylated anti-TSH capture antibody. 0.08 mL TSH antigen (10 μlU/mL ELISA calibrator) was diluted to 0.0195 μlU/mL in 41 mL PBS buffer below the Functional Sensitivity (<0.054 μlU/mL) of the DRG TSH Ultrasensitive ELISA (Part No. EIA-1790, Lot No. RN58849), and 1 mL was saved as the Baseline Sample (prior to enrichment). The 40 mL sample was processed using a VERAPREP Concentrate TSH protocol to produce a 1.0 mL Enriched Sample for subsequent TSH ELISA testing:

Dilute 80 μL of a 10 μlU/mL TSH standard to 0.0195 μlU/mL in 41.0 mL PBS, and save 1.0 mL as a Baseline Sample (prior to enrichment)
1. Dilute 80 μL of a 10 μlU/mL TSH standard to 0.80 μlU/mL in 1.0 mL VERAPREP Cleave as Control
2. Add 40 mL of 0.0195 μlU/mL TSH in PBS to a 50 mL Falcon Tube
3. Add VERAPREP Concentrate TSH, mix
4. Incubate 60 min at Room Temperature with mixing
5. Magnetically separate VERAPREP Concentrate TSH for 60 min using the Dexter LifeSep® 50SX
6. Decant and discard the 40 mL PBS into waste
7. Add 4.0 mL PBS Wash Buffer, mix
8. Magnetically separate VERAPREP Concentrate TSH in 4 mL PBS Wash Buffer for 30 min using the Dexter LifeSep® 50SX.
9. Decant and discard the 4 mL PBS into waste
10. Add 1 mL PBS Wash Buffer, mix
11. Transfer 1 mL VERAPREP Concentrate TSH to a 1.75 mL conical bottom snap-cap vial
12. Magnetically separate VERAPREP Concentrate TSH in 1 mL PBS Wash Buffer for 10 min using the Dexter LifeSep® 1.5S.
13. Aspirate and discard the 1 mL PBS into waste
14. Add 1 mL VERAPREP Cleave, mix
15. Magnetically separate VERAPREP Concentrate TSH in 1 mL VERAPREP Cleave for 10 min using the Dexter LifeSep® 1.5S.
16. Aspirate and save 1 mL supernatant (Enriched Sample), and test the Control, Baseline Sample and Enriched Sample.

0.08 mL TSH antigen (10 μlU/mL ELISA calibrator) was also diluted to 0.800 μlU/mL in 1 mL in VERAPREP Cleave buffer as the Control. The Baseline Sample, Enriched Sample, and Control were tested by the DRG TSH Ultrasensitive ELISA, and TSH % Recovery of the Enriched Sample was calculated as [Enriched Sample result]/[Control result]×100%. As expected, the diluted TSH Baseline Sample was undetectable by the Ultrasensitive ELISA and read 0.00 μlU/mL. Using only 0.80 mg reagent, VERAPREP Concentrate TSH successfully enriched the diluted TSH from undetectable to 0.73 μlU/mL (Table 2). As compared to the Control this was a 98.6% recovery, but there may have been a matrix effect of the VERAPREP Cleave buffer in the TSH ELISA that suppressed assay signal (Table 3).

TABLE 2

| | Theoretical TSH Result | Oberved TSH Result |
|---|---|---|
| Sample | (μIU/mL) | (μIU/mL) |
| Control | 0.8000 | 0.74 |
| Baseline Sample | 0.0195 | 0.00 |
| Enriched Sample | 0.7800 | 0.73 |

TABLE 3
% TSH Recovery

([Enriched Sample]/[Control]) × 100%
([0.73 μIU/mL]/[0.74 μIU/mL) × 100% = 98.6%

In the second study, VERAPREP Concentrate PTH reagent was prepared by coating 550 nm VERAPREP Biotin with biotinylated anti-PTH capture antibody. 0.021 mL PTH antigen (971 pg/mL ELISA calibrator) was diluted to 0.497 pg/mL in 41 mL PBS buffer below the Functional Sensitivity (<1.56 pg/mL) of the DRG PTH (Parathyroid) Intact ELISA (Part No. EIA-3645, Lot No. 2896), and 1 mL was saved as the Baseline Sample (prior to enrichment). The 40 mL sample was processed using the VERAPREP Concentrate PTH protocol to produce a 1.0 mL Enriched Sample for subsequent PTH ELISA testing:
1. Dilute 21 μL of a 971 pg/mL PTH standard to 0.497 pg/mL in 41.0 mL PBS, and save 1.0 mL as a Baseline Sample (prior to enrichment)
2. Dilute 21 μL of a 971 pg/mL PTH standard to 20.4 pg/mL in 1.0 mL VERAPREP Cleave as Control
3. Add 40 mL of 0.497 pg/mL PTH in PBS to a 50 mL Falcon Tube
4. Add VERAPREP Concentrate PTH, mix
5. Incubate 30 min at Room Temperature with mixing
6. Magnetically separate VERAPREP Concentrate PTH for 15 min using the Dexter LifeSep® 50SX
7. Decant and discard the 40 mL PBS into waste
8. Add 4.0 mL PBS Wash Buffer, mix
9. Magnetically separate VERAPREP Concentrate PTH in 4 mL PBS Wash Buffer for 10 min using the Dexter LifeSep® 50SX.
10. Decant and discard the 4 mL PBS into waste
11. Add 1 mL PBS Wash Buffer, mix
12. Transfer 1 mL VERAPREP Concentrate PTH to a 1.75 mL conical bottom snap-cap vial
13. Magnetically separate VERAPREP Concentrate PTH in 1 mL PBS Wash Buffer for 10 min using the Dexter LifeSep® 1.5S.
14. Aspirate and discard the 1 mL PBS into waste
15. Add 1 mL VERAPREP Cleave, mix
16. Magnetically separate VERAPREP Concentrate PTH in 1 mL VERAPREP Cleave for 10 min using the Dexter LifeSep® 1.5S.
17. Aspirate and save 1 mL supernatant (Enriched Sample), and test the Control, Baseline Sample and Enriched Sample.

0.021 mL PTH antigen (971 pg/mL ELISA calibrator) was also diluted to 20.4 pg/mL in 1 mL in VERAPREP Cleave buffer as the Control. The Baseline Sample, Enriched Sample, and Control were tested by the DRG PTH (Parathyroid) Intact ELISA, and PTH % Recovery of the Enriched Sample was calculated as [Enriched Sample result]/[Control result]×100%. The diluted PTH Baseline Sample read 13.5 pg/mL due to a matrix effect of the VERAPREP Cleave buffer in the ELISA assay. This matrix effect resulted in enhanced assay signal. Using only 0.80 mg reagent, VERAPREP Concentrate PTH successfully enriched the diluted PTH to 42.3 pg/mL (Table 4). As compared to the Control this was a 109% recovery (Table 5).

TABLE 4

| | Theoretical PTH Result | Oberved PTH Result |
|---|---|---|
| Sample | (pg/ml) | (pg/mL)) |
| Control | 20.4 | 38.8 |
| Baseline Sample | 0.497 | 13.5 |
| Enriched Sample | 20.0 | 42.3 |

TABLE 5
% PTH Recovery

([Enriched Sample]/[Control]) × 100%
([42.3 pg/mL]/[38.8 pg/mL) × 100% = 109%

### Example 4: Low Abundance Biomarker Enrichment from Urine for Subsequent Mass Spectrometry (LC-MS/MS or MALDI-MS) Analysis

The following describes a Mass Spectrometry sample pre-treatment protocol to enrich a low abundance biomarker and spiked internal standard (ISTD) from a large volume urine sample using superparamagnetic nanoparticles coated with a capture moiety specific for the biomarker. The exact same protocol could also use a plurality of different superparamagnetic nanoparticles populations mixed together or pooled together, where each population is coated with a different capture moiety, in order to multiplex and enrich more than 1 biomarker and corresponding spiked ISTD from the same sample. The enrichment and characterization of 2 or more biomarkers facilitates the use of an algorithm for the clinical diagnosis and/or prognosis of disease that is not possible with the characterization of a single biomarker. For example, for the diagnosis of obstructive sleep apnea (OSA) from urine, the VERAPREP Concentrate reagent could comprise 4 different antibodies to capture and enrich kallikrein-1, uromodulin, urocortin-3 and orosomucoid-1, or 7 different antibodies to capture and enrich kallikrein-1, uromodulin, urocortin-3 and orosomucoid-1, IL-6, IL-10 and high sensitivity C-reactive protein:
1. Collect patient urine (use standard urine collection protocol such as a urine collection cup)
2. Mix urine collection sample
3. Add 40 mL urine to the 50 mL Falcon Tube
4. Add Deuterated Internal Standard for the biomarker to be enriched, mix
5. Add VERAPREP Condition, mix
6. Add VERAPREP Concentrate, mix
7. Incubate: biomarker+deuterated internal standard capture by VERAPREP Concentrate
8. Magnetically separate VERAPREP Concentrate in the 40 mL urine using the Dexter LifeSep® 50SX
9. Aspirate and discard the urine into waste
10. Add 4 mL PBS Wash Buffer, mix
11. Magnetically separate VERAPREP Concentrate in 4 mL PBS Wash Buffer using the Dexter LifeSep® 50SX.
12. Aspirate and discard the urine into waste
13. Repeat Step 12 two more times (2×)
14. Add 1 mL VERAPREP Cleave, mix (Mass Spectrometry compatible buffer)
15. Magnetically separate VERAPREP Concentrate in 1 mL VERAPREP Cleave using the Dexter LifeSep® 1.5S.
16. Aspirate and Test the 1 mL supernatant sample by LC-MS
17. Final biomarker concentration determined based on: 1) 40 mL urine sample size, 2) LC-MS quantitation of the biomarker, and 3) adjust reported biomarker value based on Deuterated Internal Standard recovery

The selective release or cleavage of the captured and enriched biomarker or biomarkers can be accomplished with a change in pH (acidic pH such as glycine pH 2.5 elution followed by neutralization, or alkaline pH 10.0 or greater), using a cleavable linker such as a disulfide bond cleaved with a reducing agent such as TCEP or DTT, or by using competitive elution such a molar excess of D-biotin with monomeric avidin or molar excess of sugar with Concanavalin A that compete for the binding sites on Concanavalin A.

### Example 5. SARS-CoV-2 Neutralizing Antibody Assay

This assay isolates and quantitates SARS-CoV-2 neutralizing antibody recognizing both the receptor binding domain and the N-terminal domain. It quantitates IgG, IgA, and IgM separately. As carried out below, serum antibody was assayed, but the procedure can also be carried out on oral saline rinse to assay saliva antibody. In the assay, sample is first cleaned to remove potential heterophilic interferences, antibody is captured on beads (microparticles) and reacted with a detection reagent, the captured antibody (still bound by the detection reagent) is eluted from the bead, and the supernatant transferred for quantitation. The assay can be carried out manually or automated. Generalized procedures for the assay include:

1. Prime plate washer with wash/blocking buffer
a. Sonicate for 30 min with water first.

2. Prepare all reagents
a. All beads should be mixed and on a rocker such that they are homogenous for use

3. Plate conditioning—a pair of clear, round bottom 96-well microtiter plates are washed with a wash buffer of 0.023% (w/v) Pluronic® F108 (poly(ethylene glycol)-block-poly(propylene glycol)-block-poly(ethylene glycol), a triblock copoloymer) in TTA (Tris-buffered saline, 0.05% Tween® 20 (polysorbate 20), 0.05% azide, pH 7.4) to block non-specific binding, for example, of the immunoglobulins, to the well surfaces. One round bottom plate will serve as a “cleaning” plate and the other as a “capture” plate.

4. Pre-analytic sample cleaning to remove heterophilic interferences.
a. Use samples directly from refrigerated storage, without mixing of centrifugation. Avoid directly pipetting any lipids.
b. Add 60 μL of each sample in the “Clean” Plate layout into the blocked round bottom plate
c. Once all samples are added, use a multichannel pipette to add 140 μL of clean beads (mixture of 36 μg Rabbit IgG-biotin-Streptavidin beads and 4 μg Human IgG-biotin-Streptavidin beads to capture and remove heterophilic interference specific to rabbit IgG, human IgG, streptavidin, and/or the beads themselves) to all wells with samples.
   i. Note: the base 1.6 micron superparamagnetic magnetic streptavidin beads used in Clean Beads are the exact same base 1.6 micron superparamagnetic streptavidin beads used in the Capture Beads. This way any heterophilic interference specific to the base Streptavidin Beads is removed from the sample prior to testing the clean sample with the Capture Beads.
d. Incubate 15 min at 37° C. with shaking in plate washer
   i. Orbital shaking at fast setting, 425 cpm
e. Place on magnet for 4 min.
   i. for example, Alpaqua Catalyst 96, Part Number A000550.

5. Capture neutralizing antibodies for detection and multiplex quantitation of IgM, IgG, and IgA specific for SARS-CoV-2 S1-RBD or S1-NTD.
a. Transfer 50 μL of the cleaned samples into the blocked capture plate (clear flat bottom) using a multichannel pipette.
b. Add 150 μL of capture beads (mixture of 30 μg SARS-CoV-2 S1-RBD-biotin-Streptavidin beads and 10 μg SARS-CoV-2 S1-NTD-biotin-Streptavidin beads used to capture human immunoglobulins IgA, IgG and/or IgM specific to RBD or NTD) to each well using a multichannel pipette.
   i. Add same volume of capture beads to an empty well to serve as a capture bead blank.
   ii. SARS-CoV-2 Spike Glycoprotein (S1) RBD (with His-tag and produced in HEK293; The Native Antigen Company, Part. No. REC31882).
   iii. SARS-CoV-2 Spike NTD (with His-tag and produced in HEK293; The Native Antigen Company, Part. No. REC31905).
c. Add 60 μL of triplex calibrator beads to six wells of the plate, each containing a different calibrator level (see below).
   i. Set plate on magnet while adding triplex calibrator beads and “rinse” the tip as you dispense each calibrator.
d. Incubate 30 min at 37° C. with shaking in the plate reader.
   i. Orbital shaking at fast setting, 425 cpm.
e. wash 3× on plate washer.
   i. 2 min initial/shake steps.

6. Multiplex labeling of captured antibody.
a. Add 200 μL Triplex conjugate per well.
   i. The same set of 8 tips on a multi-channel pipette can be used to add the conjugate to all wells across the plate
   ii. Once all wells have conjugate, go back and pipette up and down 5× to thoroughly mix wells (use multi-channel pipette but get fresh tips for each column).
   iii. The Triplex conjugate is 0.002 mg/ml polyclonal rabbit anti-human IgM conjugated with AlexaFluor-488, 0.002 mg/ml polyclonal rabbit anti-human IgG conjugated with AlexaFluor-555, and 0.002 mg/ml polyclonal rabbit anti-human IgA conjugated with AlexaFluor-647, in Conjugate buffer (0.1% BSA in TTA).
b. Incubate 30 min at 37C with shaking in the plate reader.
   i. Orbital shaking at fast setting, 425 cpm.
c. wash 3× on plate washer.
   i. 2 min initial/shake steps.

7. Elution of antibody-conjugate complex from bead
a. Pre-fill wells on a clear bottom, black plate (not blocked) with 35.5 MI of Neutralization buffer (300 mM Tris pH 10.0).
b. Take capture plate off plate washer.
c. Add 220 μL Elution Buffer (100 mM Glycine, pH 2.5).
   i. The same set of 8 tips on a multi-channel pipette can be used to add the conjugate to all wells across the plate.
   ii. Once all wells have elution buffer, go back and pipette up and down 5× to thoroughly mix wells (use multi-channel pipette but get fresh tips for each column).
d. Place on magnet for 2 min.
e. Transfer 200 uL of each well to the black, clear flat bottom read plate (of step 7a) using multi-channel pipette.

8. Read fluorescence on plate reader.

Triplex calibrator beads are assembled from four components:
1.6 μm magnetic beads covalently modified with streptavidin and reacted with biotinylated human IgA (affinity purified),
1.6 μm magnetic beads covalently modified with streptavidin and reacted with biotinylated human IgG (affinity purified),
1.6 μm magnetic beads covalently modified with streptavidin and reacted with biotinylated human IgM (affinity purified),
1.6 μm magnetic beads (carboxyl) quenched with Tris (TRis beads), each stored at 10 mg/ml is a Calibrator Bead Storage solution of 0.1% BSA in TTA.

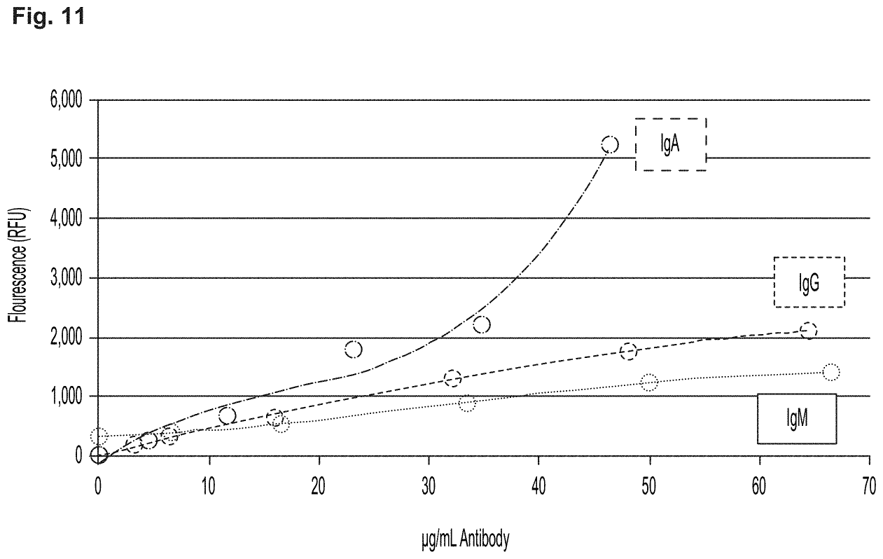

The beads are diluted to 1.00 mg/ml with Calibrator Bead Storage solution and the IgA, IgG, and IgM beads each separately pooled with Tris beads in the following ratios: 100:0, 75:25, 50:50, 25:75, 10:90, and 0:100. For each non-zero Ig calibration level, the IgA, IgG, and IgM mixtures are then pooled 1:1:1 to create a set triplex calibrator beads. The triplex calibrator beads are used at a final concentration of 0.3 mg/ml in Calibrator Bead Storage solution. A calibration curve is shown in FIG. 11.

Following the protocol essentially as described above, a 146 serum samples collected before December 2018 we tested for the presence of SARS-CoV-2 neutralizing antibody. As this was well before the virus emerged in the human populations the samples would be expected to be negative, and indeed this is what was found (see Table 6).

TABLE 6
Total SARS-CoV-2 Antibody (IgA, IgG, and IgM) - Specificity

| | No. | No. | No. | Specificity, % | |
|---|---|---|---|---|---|
| Group | Tested | Nonreactive | Reactive | (95% CI*) | |
| Diagnostic | 96 | 95 | 1 | 98.96 | (93.51-99.95) |
| Routine | | | | | |
| Blood | 50 | 50 | 0 | 100 | (91.11-100.0) |
| donors | | | | | |
| Overall | 146 | 145 | 1 | 99.32 | (95.67-99.96) |

*Confidence Interval

“Diagnostic routine” refers to serum samples remaining after routine diagnostic testing, where the health status of the donor is not known. “Blood donors” refers to samples saved from blood donations from healthy donors. These results show that the assay meets the FDA emergency use authorization Specificity requirement of 95%.

The assay was also used to test 122 samples for 63 symptomatic patients with PCR-confirmed SARS-CoV-2 infection. These samples included one or more consecutive specimens collected from the date of symptom onset. The results are presented in Table 7.

TABLE 7
Total SARS-CoV-2 Antibody (IgA, IgG, and IgM) - Sensitivity

| Days post | | | | | |
|---|---|---|---|---|---|
| symptom | No. | No. | No. | Sensitivity, % | |
| onset | Tested | Reactive | Nonreactive | (95% CI*) | |
| 0-6 | 5 | 2 | 3 | 40.00 | (7.26-82.96) |
| 7-13 | 13 | 12 | 1 | 92.31 | (62.09-99.60) |
| ≥14 | 103 | 100 | 3 | 97.09 | (91.10-99.24) |

| Days post | | | | |
|---|---|---|---|---|
| PCR swab | No. | No. | No. | Sensitivity, % |
| collection | Tested | Reactive | Nonreactive | (95% CI*) |
| 0-6 | 13 | 9 | 4 | 69.23 (33.88-89.64) |
| 7-13 | 20 | 19 | 1 | 95.00 (73.06-99.74) |
| ≥14 | 89 | 87 | 2 | 97.75 (91.35-99.61) |

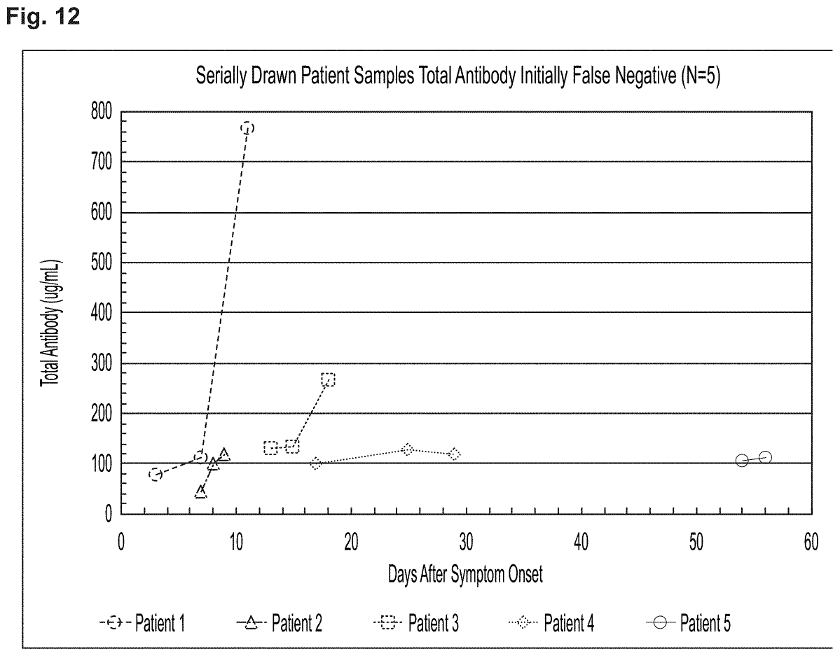

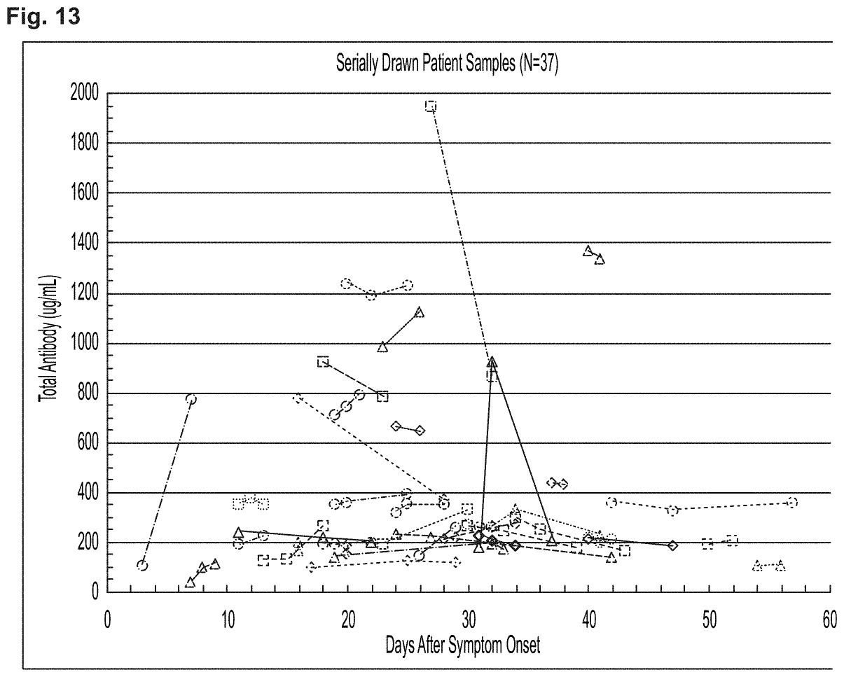

These results show that the assay meets the FDA emergency use authorization Sensitivity requirement of 90%. Several samples giving false negative results in the initial assay gave positive results for later collected samples (FIG. 12). While many patients from which there were 2 or 3 consecutive samples showed similar levels of antibody over time, some patients showed rapidly increasing or decreasing antibody levels (FIG. 13).

### Example 6. Cleaning and Capturing Biomarker from a Saline Oral Rinse

To clean 1 mL (1000 μL) of saline oral rinse sample (5 mL 0.9% NaCl swished for 25 seconds, gargled for 5 seconds, and expectorated into a collection tube=5 mL saline+saliva sample, or a saliva-based sample in saline), add either 2× or 4× clean beads with the following composition:

For 2× clean beads per test:
25 ug Rabbit IgG beads
25 ug BSA beads
10 ug purified human IgA beads
10 ug purified human IgG beads
10 ug purified human IgM beads

For 4× clean beads per test:
50 ug Rabbit IgG beads
50 ug BSA beads
20 ug purified human IgA beads
20 ug purified human IgG beads
20 ug purified human IgM beads

To capture the SARS-CoV-2 neutralizing antibodies from a cleaned 1 mL saline oral rinse sample, we add 5× or 10× capture beads to enrich and capture total neutralizing immunoglobulins from the clean saline oral rinse samples with this amount of RBD and NTD capture beads:

For 5× capture beads per test:
150 ug RBD beads
50 ug NTD beads

For 10× capture beads per test:
300 ug RBD beads
100 ug NTD beads

Results of an assay using 10× clean beads and 10× capture beads on three patients having received one administration (of the usual two) of a SARS-CoV-2 mRNA vaccine and 3 unvaccinated subjects are shown in Table 8. These data were collected only 8 days after administration and show that even at this early time-point the assay is able to detect (and quantitate) a SARS-CoV-2 neutralizing antibody response. (A person is generally not considered “fully vaccinated” until two weeks after a 2nd administration of the vaccine.)

TABLE 8
Antibody detected in oral saline rinse.

| | | Neutralizing Antibodies (μg/ml) | | |
|---|---|---|---|---|
| | mRNA Vaccine | IgA | IgG | IgM |
| Patient 1 | 1 shot | 13.1 | 11.4 | 6.3 |
| Patient 2 | 1 shot | 34.3 | 11.2 | 10.2 |
| Patient 3 | 1 shot | 10.9 | 34.6 | 6.5 |
| Patient 4 | None | 4.4 | 1.7 | 5.7 |
| Patient 5 | None | 13.8 | 4.4 | 8.3 |
| Patient 6 | None | 13.5 | 0.0 | 6.8 |

As can be seen, patient 2 has above background levels of SARS-CoV-2 specific antibody of all three isotypes and all three vaccinated patients have above background levels of SARS-CoV-2 specific IgG.

### ABBREVIATIONS

ABEI N-(4-aminobutyl)-N-ethylisoluminol

ALP Alkaline phosphatase

BSA Bovine serum albumin

Fab Fragment antibody-binding

Fc Fragment, crystallizable

HAAA Human anti-animal antibody

HAMA Human anti-mouse antibody

HASA Human anti-sheep antibody

IFU Instructions for use

IgG Antibody or immunoglobulin

IgM Immunoglobulin M

HRP Horse radish peroxidase

LC-MS/MS Liquid chromatography tandem-mass spectrometry

LDT Laboratory developed test

Mab Monoclonal antibody

MASI Manufacture assay specific interference

MFG IVD Manufacturers

PMP Superparamagnetic microparticles

PBCT Primary blood collection tubes

RF Rheumatoid Factor

RLU Relative light units or assay response signal

RUO Research use only

SAv Streptavidin

STT Secondary transfer tubes

TAT Turnaround time

WF Work flow

### DEFINITIONS

As used herein, “sample” or “biological sample” refers to any human or animal serum, plasma (i.e. EDTA, lithium heparin, sodium citrate), blood, whole blood, processed blood, urine, saliva, stool (liquid and solid), semen or seminal fluid, amniotic fluid, cerebral spinal fluid, cells, tissues, biopsy material, DNA, RNA, or any fluid, dissolved solid, or processed solid material to be tested for diagnosis, prognosis, screening, risk assessment, risk stratification, and monitoring such as therapeutic drug monitoring. In some embodiments, the sample is a large volume sample. In some embodiments, the sample comprises a plurality of samples (e.g., more than one sample from the same or a different subject. In some embodiments, the sample comprises a biomarker present at low abundance in the sample.

In some embodiments, the sample is collected into in a primary blood collection tube (PBCT), secondary transfer tube (SST), blood collection bag, 24-hour (24-hr) urine collection device, vericore tubes, nanotainer, a saliva collection tube, blood spot filter paper, or any collection tube or device such as for stool and seminal fluid, a light green top or green top plasma separator tube (PST) containing sodium heparin, lithium heparin or ammonium heparin, a light blue top tube containing sodium citrate (i.e. 3.2% or 3.8%) or citrate, theophylline, adenosine, dipyridamole (CTAD), a red top tube for Serology or Immunohematology for the collection of serum in a glass (no additives) or plastic tube (contains clot activators), a red top tube for Chemistry for the collection of serum in a glass (no additives) or plastic tube (contains clot activators), a purple lavender top tube containing EDTA K2, EDTA K3, liquid EDTA solution (i.e. 8%), or EDTA K2/gel tubes for testing plasma in molecular diagnostics and viral load detection, a pink top tube for Blood Bank EDTA, a gray top tube containing potassium oxalate and sodium fluoride, sodium fluoride/EDTA, or sodium fluoride (no anticoagulant, will result in a serum sample), a yellow top tube containing ACD solution A or ACD solution B, a royal blue top (serum, no additive or sodium heparin), a white top tube, or any color or tube type, for any application or diagnostic test type, containing no additives or any additive or combinations thereof, for the collection of blood.

In some embodiments, the sample is a challenging sample type such as urine, 24-hour urine, saliva and stool, or where a biomarker of interest may be dilute or difficult to measure. For example, the biological sample can be a challenging because of the patient population (e.g., neonatal, pediatric, geriatric, pregnant, oncology, autoimmune disease). For example, some biomarkers are too dilute or at too low of concentration, e.g., in circulation, or in urine, to be reliably detected and accurately and precisely measured by existing POCT and central laboratory analyzers. In some embodiments, the challenging sample is cerebrospinal fluid (CSF).

As used herein, “collection device” can be a primary blood collection tube (PBCT), 24-hr urine collection device, a urine collection device, a saliva collection tube, a stool collection device, a seminal fluid collection device, a blood collection bag, or any sample collection tube or device, prior to the addition of the sample.

A PBCT and secondary transfer tube (SST) can be any commercially available standard or custom collection tube (with or without gel separators) from companies like Becton Dickinson (BD), Greiner, VWR, and Sigma Aldrich, a glass tube, a plastic tube, a light green top or green top plasma separator tube (PST) containing sodium heparin, lithium heparin or ammonium heparin, light blue top tube containing sodium citrate (i.e. 3.2% or 3.8%) or citrate, theophylline, adenosine, dipyridamole (CTAD), red top tube for Serology or Immunohematology for the collection of serum in a glass (no additives) or plastic tube (contains clot activators), a red top tube for Chemistry for the collection of serum in a glass (no additives) or plastic tube (contains clot activators), a purple lavender top tube containing EDTA K2, EDTA K3, liquid EDTA solution (i.e. 8%), or EDTA K2/gel tubes for testing plasma in molecular diagnostics and viral load detection, a pink top tube for Blood Bank EDTA, a gray top tube containing potassium oxalate and sodium fluoride, sodium fluoride/EDTA, or sodium fluoride (no anticoagulant, will result in a serum sample), a yellow top tube containing ACD solution A or ACD solution B, a royal blue top (serum, no additive or sodium heparin), a white top tube, or any color or tube type, for any application or diagnostic test type, containing no additives or any additive or combinations thereof, for the collection of blood.

As used herein, a “storage device” or “transfer device” refers to a device that receives the sample and/or other components received in a collection device. The storage or transfer device can be a plastic or glass tube, vial, bottle, beaker, flask, bag (e.g., a blood collection bag, can, microtiter plate, ELISA plate, 96- well plate, 384-well plate 1536 well plate, cuvette, reaction module, reservoir, or any container suitable to hold, store or process a liquid sample.

As referred to herein, a “diagnostic test” includes, but is not limited to any antibody-based diagnostic test, non-antibody based diagnostic test, a sample pre-treatment method or device for subsequent analysis by chromatographic, spectrophotometric, and mass spectrometry methods (i.e. HPLC, MS, LCMS, LC-MS/MS) such as immunoextraction (IE) and solid phase extraction (SPE), radioimmunoassay (RIA), enzyme-linked immunoassay (ELISA), chemiluminescence immunoassay (CLIA), molecular diagnostics, lateral flow (LF), point-of-care (PoC), direct to consumer (DTC), CLIA and CLIA waived tests and devices, Research Use Only (RUO) test, In Vitro Diagnostics (IVD) test, Laboratory Developed Test (LDT), companion diagnostic, and any test for diagnosis, prognosis, screening, risk assessment, risk stratification, and monitoring such as therapeutic drug monitoring. In some embodiments, the diagnostic test comprises short turn-around time (STAT) diagnostic tests, ambulatory tests, lateral flow tests, point of care (PoC) tests, molecular diagnostic tests, HPLC, MS, LCMS, LC-MS/MS, radioimmunoassay (RIA), enzyme-linked immunoassay (ELISA), chemiluminescence immunoassay (CLIA), CLIA and CLIA waived tests, and any diagnostic test used for the diagnosis, prognosis, screening, risk assessment, risk stratification, treatment monitoring, and therapeutic drug monitoring.

As used herein, a pathogen is a bacterium, virus, or other microorganism that can cause disease.

Serology is the scientific study of serum and other body fluids. In practice, the term usually refers to the diagnostic identification of antibodies in serum. Such antibodies are typically formed in response to an infection (against a given microorganism), against other foreign proteins (in response, for example, to a mismatched blood transfusion), or to one's own proteins (in instances of autoimmune disease).

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method for isolating an antigen-specific antibody from a biological sample, the method comprising:
a) combining the sample with a first particle comprising a capture moiety for the antigen-specific antibody to provide a mixture;
b) mixing the mixture to provide particle complexes to the antibody; and
c) separating the particle from the biological sample;
thereby isolating the antibody from the biological sample.

2. The method of claim 1, further comprising dissociating the antigen-specific antibody from the particle.

3. The method of claim 1, wherein dissociating comprises cleavage or elution of the antibody from the first particle.

4. The method of claim 3, wherein the method further comprises subjecting the released antibody to characterization.

5. The method of claim 4, wherein characterization comprises forming a complex with an anti-immunoglobulin antibody conjugated to a detectable label.

6. The method of claim 5, wherein the detectable label is a fluorescent label.

7. The method of claim 4, further comprising comparison of a signal associated with the antigen-specific antibody with a standard curve for immunoglobulin.

8. The method of claim 5, wherein the anti-immunoglobulin antibody is not isotype specific.

9. The method of claim 5, wherein the anti-immunoglobulin antibody is isotype specific.

10. The method of claim 9, wherein the isotype-specific anti-immunoglobulin antibody comprises at least two of anti-IgA, anti-IgG, and anti-IgM, each conjugated to a distinct label.

11. The method of claim 1, further comprising a pretreatment, the pretreatment comprising
a) combining a biological sample with a second particle comprising a capture moiety for an interference to provide a mixture;
b) mixing the mixture to provide second particle complexes to the interference;
c) removing or eliminating the second particle complexes to provide a depleted solution.

12. The method of clam 11, wherein the capture moiety comprises human and/or non-human animal immunoglobulin.

13. The method of claim 11, wherein the capture moiety comprises streptavidin.

14. The method of claim 1, wherein the first and/or second particle is provided as a lyophilized product.

15. An enriched antibody, made by the method of claim 1.

16. The method of claim 1, wherein the antigen-specific antibody is a pathogen-specific antibody.

17. The method of claim 16, wherein the pathogen is SARS-CoV-2.

18. The method of claim 17, wherein the capture moiety is a spike protein of SARS-CoV-2.

19. The method of claim 18, wherein the spike protein is the S1 subunit, or a receptor binding domain and/or an N-terminal domain thereof.

20. The method of claim 1, wherein the antigen-specific antibody is an autoantibody.

21. The method of claim 20, wherein the antigen-specific antibody is a tumor antigen-specific autoantibody.

22. The method of claim 1, wherein the antigen-specific antibody is against a foreign protein.

23. The method of claim 1, wherein the antigen-specific antibody is a human antibody.
